SEQUENCE LISTING (5 sequences)

SEQ ID NO: 1 - protein, 371 aa, Homo sapiens
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 2 - DNA, 1579 bp, Homo sapiens
aattcggcacgagggcatggggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaattatggagagtgaagaagtttctcattcccagcgtgccagacccgaaatccatcttccccgggctctttgagatacaccaagggaacttccaggagtggatcacagacacccagaacgtggcccacctccacaagatggcaggtgcagagcaagaaagtggccccgaggagcccctggtagtccagttggccaagactgaagccgagtctcccaggatgctggacccacagaccgaggagaaagaggcctctgggggatccctccagcttccccaccagcccctccaaggcggtgatgtggtcacaatcgggggcttcacctttgtgatgaatgaccgctcctacgtggcgttgtgatggacacaccactgtcaaagtcaacgtcaggatccacgttgacatttaaagacagaggggactgtcccggggactccacaccaccatggatgggaagtctccacgccaatgatggtaggactaggagactctgaagacccagcctcaccgcctaatgcggccactgccctgctaactttcccccacatgagtctctgtgttcaaaggcttgatggcagatgggagccaattgctccaggagatttactcccagttccttttcgtgcctgaacgttgtcacataaaccccaaggcagcacgtccaaaatgctgtaaaaccatcttcccactctgtgagtccccagttccgtccatgtacctgttccatagcattggattctcggaggattttttgtctgttttgagactccaaaccacctctacccctacaaaaaaaaaaaaaaaaaa

SEQ ID NO: 3 - protein, 371 aa, Artificial sequence
  F232C mutant of human CRLF2 polypeptide
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKCILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 4 - DNA, 1116 bp, Artificial sequence
  Nucleic acid encoding mutant human CRLF2 polypeptide
atggggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatgtattttaatttccagcctggccatccttctgatggtgtctctcctccttctgtctttatggaaattatggagagtgaagaagtttctcattcccagcgtgccagacccgaaatccatcttccccgggctctttgagatacaccaagggaacttccaggagtggatcacagacacccagaacgtggcccacctccacaagatggcaggtgcagagcaagaaagtggccccgaggagcccctggtagtccagttggccaagactgaagccgagtctcccaggatgctggacccacagaccgaggagaaagaggcctctgggggatccctccagcttccccaccagcccctccaaggcggtgatgtggtcacaatcgggggcttcacctttgtgatgaatgaccgctcctacgtggcgttgtga

SEQ ID NO: 5 - DNA, 1116 bp, Homo sapiens
atggggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaattatggagagtgaagaagtttctcattcccagcgtgccagacccgaaatccatcttccccgggctctttgagatacaccaagggaacttccaggagtggatcacagacacccagaacgtggcccacctccacaagatggcaggtgcagagcaagaaagtggccccgaggagcccctggtagtccagttggccaagactgaagccgagtctcccaggatgctggacccacagaccgaggagaaagaggcctctgggggatccctccagcttccccaccagcccctccaaggcggtgatgtggtcacaatcgggggcttcacctttgtgatgaatgaccgctcctacgtggcgttgtga

(19) United States
(12) Patent Application Publication
(10) Pub. No.: US 2012/0282258 A1
(43) Pub. Date: Nov. 8, 2012

(54) CRLF2 IN PRECURSOR B-CELL ACUTE LYMPHOBLASTIC LEUKEMIA

(75) Inventors: David Weinstock, Jamaica Plain, MA (US); Akinori Yoda, Brookline, MA (US)

(73) Assignee: DANA-FARBER CANCER INSTITUTE, INC., Boston, MA (US)

(21) Appl. No.: 13/508,395

(22) PCT Filed: Nov. 24, 2010
(86) PCT No.: PCT/US10/57942

§ 371 Date: Jul. 27, 2012

Related U.S. Application Data
(60) Provisional application No. 61/264,315, filed on Nov. 25, 2009.

(51) Int. Cl. C07K 16/30; C12N 15/12; C12N 15/63; C12N 5/10; A61P 35/02; A61K 31/7088; C12Q 1/68; C07K 14/705; A61K 39/395

(52) U.S. Cl. 424/138.1; 530/350; 536/23.5; 435/320.1; 530/387.7; 424/178.1; 514/44 A; 435/6.12; 530/391.7; 435/369; 435/358; 435/325

(57) ABSTRACT

The invention relates to cytokine receptor-like factor 2 (CRLF2), and particularly certain mutant forms of CRLF2, as prognostic and therapeutic targets in precursor B-cell acute lymphoblastic leukemia (B-ALL). Mutant CRLF2 with a Phe232-Cys (F232C) mutation is overexpressed and constitutively activates STAT5 in a subset of B-ALL patients with particularly poor prognosis. Methods and compositions useful for identifying, inhibiting expression, and inhibiting activity of the mutant CRLF2 are provided. Also provided are methods and compositions useful for treating B-ALL.

### BACKGROUND OF THE INVENTION

The majority of adults with precursor B-cell acute lymphoblastic leukemia (B-ALL) will die from their disease. Over the past decade, studies using oligonucleotide arrays and high-throughput sequencing identified several genetic and transcriptional aberrations in B-ALL, leading to three conceptual advances. First, genes involved in normal B-cell development (e.g., PAX5, IKZF1) are frequently mutated in B-ALL. Second, B-ALL is highly heterogeneous and can exist as multiple, genetically-distinct clones within the same individual. Third, B-ALL transcriptional profiles cluster based on characteristic chromosomal rearrangements, particularly rearrangements of TEL, MLL, TCF3, and BCR/ABL.

However, one-third of B-ALL lack characteristic rearrangements. Faderl S et al. (1998) Blood 91:3995-4019. Transcriptional profiles from a subset of these leukemias cluster with profiles from BCR/ABL-expressing B-ALL, suggesting that the former harbor cryptic alterations in tyrosine kinase signaling. Supporting this notion, mutations in Janus kinases (JAKs) were recently identified in a small percentage of pediatric B-ALL, but approximately 20% of ALL in children with Down Syndrome.

In addition, IGH translocations involving the pseudoautosomal region 1 (PAR1) of both sex chromosomes have been reported to dysregulate the cytokine receptor-like factor 2 (CRLF2) gene in a subset of patients with B-ALL. Russell L J et al. (2009) Blood 114:2688-98. Whereas normally CRLF2 is not expressed on mature B cells, and CRLF2 may be expressed in normal early B cells, expression levels of CRLF2 in B-ALL patients with the translocation was reported to be hundreds to thousands of times higher than in B-ALL patients without the translocation. B-ALL patients with CRLF2 overexpression were also reported to have constitutive phosphorylation of Janus kinase 2 (JAK2) and signal transducer and activator of transcription 5 (STAT5).

CRLF2, which is also known as CRL2, thymic stromal-derived lymphopoietin receptor, and thymic stromal lymphopoietin receptor (TSLPR), is a 371-amino acid type I transmembrane protein which, when normally expressed as a heterodimer in combination with the alpha chain of interleukin 7 receptor (IL-7Rα), activates STAT5 in response to interaction with thymic stromal lymphopoietin (TSLP). Normally neither CRLF2 alone nor IL-7R alone activates STAT5 in response to interaction with TSLP.

TSLP is produced by epithelial cells at sites of inflammation, where it activates myeloid dendritic cells and can stimulate T helper 2 (Th2) immune responses. TSLP has been reported also to promote early B-cell development and stimulates the growth of some human B-ALLs in vitro. Liu Y J (2009) Adv Immunol 101:1-25; Reche P A et al. (2001) J Immunol 167:336-43; Brown V I et al. (2007) Cancer Res 67:9963-70.

Recently a number of antibodies directed against CRLF2, and TSLP, have been developed for use in treating inflammatory and allergic disorders. See, for example, WO 2008/076321.

### SUMMARY OF THE INVENTION

As disclosed herein, the invention is based, in part, on the discovery by the inventors that CRLF2 Phe232Cys (F232C) and certain JAK2 Arg683 mutants are gain-of-function mutations in mutually exclusive subsets of CRLF2-overexpressing B-ALL that transform growth factor-dependent cells to factor independence. Strikingly, 100% of B-ALL with mutant JAK2 overexpress CRLF2, suggesting that CRLF2 is the essential scaffold for mutant JAK2 activity in B-ALL. The gene signature associated with CRLF2 overexpression is highly similar in both pediatric and adult cases, and significantly overlaps with a BCR/ABL signature. Together, these findings establish CRLF2 as a key factor in B-ALL, and support its use as a prognostic and therapeutic target. These findings also establish the F232C mutant form of human CRLF2 as a key factor in B-ALL, and support its use as a prognostic and therapeutic target.

An aspect of the invention is an isolated mutant human cytokine receptor-like factor 2 (CRLF2) polypeptide having an amino acid sequence at least 99% identical to SEQ ID NO:1 and comprising a F232C mutation. In one embodiment the amino acid sequence is identical to SEQ ID NO:1 except for the F232C mutation.

An aspect of the invention is an isolated nucleic acid molecule comprising a sequence that encodes the mutant human cytokine receptor-like factor 2 (CRLF2) polypeptide having an amino acid sequence at least 99% identical to SEQ ID NO:1 and comprising a F232C mutation.

An aspect of the invention is a vector comprising a nucleic acid molecule comprising a sequence that encodes the mutant human cytokine receptor-like factor 2 (CRLF2) polypeptide having an amino acid sequence at least 99% identical to SEQ ID NO:1 and comprising a F232C mutation.

An aspect of the invention is a cell comprising a vector comprising the nucleic acid molecule comprising a sequence that encodes the mutant human cytokine receptor-like factor 2 (CRLF2) polypeptide having an amino acid sequence at least 99% identical to SEQ ID NO:1 and comprising a F232C mutation.

An aspect of the invention is an isolated antibody, or antigen-binding fragment thereof, that binds specifically to the a mutant human cytokine receptor-like factor 2 (CRLF2) polypeptide having an amino acid sequence at least 99% identical to SEQ ID NO:1 and comprising a F232C mutation.

An aspect of the invention is an isolated antibody, or antigen-binding fragment thereof, that binds specifically to a homodimeric protein comprising a mutant human cytokine receptor-like factor 2 (CRLF2) polypeptide having an amino acid sequence at least 99% identical to SEQ ID NO:1 and comprising a F232C mutation.

In one embodiment, the antibody or antigen-binding fragment thereof is conjugated to a toxin.

An aspect of the invention is a method of treating precursor B-cell acute lymphoblastic leukemia (B-ALL). The method includes the step of administering to a subject having B-ALL, wherein the B-ALL is characterized by mutant human cytokine receptor-like factor 2 (CRLF2) polypeptide having an amino acid sequence at least 99% identical to SEQ ID NO:1 and comprising a F232C mutation, an effective amount of an agent that inhibits signaling by the mutant CRLF2 to treat the B-ALL.

In one embodiment, the agent comprises an antibody or antigen-binding fragment thereof that binds specifically to the mutant human CRLF2 polypeptide. In one embodiment, the antibody or antigen-binding fragment is conjugated to a toxin.

In one embodiment, the agent comprises an antisense oligonucleotide complementary to a polynucleotide encoding mutant human cytokine receptor-like factor 2 (CRLF2) polypeptide having an amino acid sequence at least 99% identical to SEQ ID NO:1 and comprising a F232C mutation.

In one embodiment, the agent comprises RNAi complementary to a polynucleotide encoding mutant human cytokine receptor-like factor 2 (CRLF2) polypeptide having an amino acid sequence at least 99% identical to SEQ ID NO:1 and comprising a F232C mutation.

In one embodiment, the method further includes administering to the subject an effective amount of a compound selected from the group consisting of JAK2 inhibitors, protein kinase C (PKC) inhibitors, heat shock protein 90 (HSP90) inhibitors, and any combination thereof.

An aspect of the invention is a method for identifying a subject at increased risk of mortality from precursor B-cell acute lymphoblastic leukemia (B-ALL). The method includes the steps of performing an assay on a sample isolated from a subject, wherein the assay detects presence of mutant human cytokine receptor-like factor 2 (CRLF2) characterized by an amino acid mutation F232C; and identifying the subject as having increased risk of mortality from B-ALL when performing the assay detects the presence of the mutant CRLF2 in the sample.

In one embodiment, the method further includes the step of detecting presence of the mutant CRLF2 in the sample.

In one embodiment, the method further includes the step of recording the result of performing the assay.

In one embodiment, the mutant CRLF2 is mutant human cytokine receptor-like factor 2 (CRLF2) polypeptide having an amino acid sequence at least 99% identical to SEQ ID NO:1 and comprising a F232C mutation.

In one embodiment, the subject has B-ALL.

An aspect of the invention is a method for identifying a subject at increased risk of mortality from precursor B-cell acute lymphoblastic leukemia (B-ALL). The method includes the step of determining the presence of mutant human cytokine receptor-like factor 2 (CRLF2) characterized by an amino acid mutation F232C by performing an assay on a sample isolated from a subject, wherein the presence of the mutant human CRLF2 in the sample indicates the subject is at increased risk of mortality from B-ALL.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1 is a cartoon depicting identification of CRLF2 in B-ALL. Tumor-derived complementary DNA (cDNA) is packaged into retroviral particles that are used to infect BaF3 cells. Integrants (gray) survive in puromycin selection. Surviving clones (black) are isolated after interleukin 3 (IL-3) withdrawal and their integrated cDNAs are sequenced and repackaged within retrovirus and confirmed.

FIG. 2 is a pair of graphs depicting disease-free survival and overall survival from the time of diagnosis for subjects having high or low/no CRLF2 expression, estimated using the Kaplan-Meier product limit method and compared in univariate analysis by the log-rank test.

FIG. 3 is a group of four graphs depicting in vitro cell growth of UT7 cells and BaF cells that stably express CRLF2 and/or JAK2 alleles grown in the absence of GM-CSF (UT7) or IL-3 (BaF3). For the bottom right panel, TSLP 1 ng/mL was added at day 0, as indicated. Growth is the number of viable cells relative to the number of cells initially seeded on day 0. Error bars represent one standard deviation.

FIG. 4 is a graph depicting bimodal distribution of CRLF2 expression in a cohort of 207 patients with high-risk pediatric B-ALL. CRLF2 overexpression is present in 29 (14.0%) of 207 cases.

FIG. 5 is a graph depicting signal activation and dependence on JAK signaling. BaF3 cells that stably express BCR/ABL, CRLF2 and/or JAK2 were grown in the absence of cytokines. +IL3 indicates unmodified BaF3 cells grown in the presence of 500 pg/mL IL-3. Data represents the ratio of viable cells exposed to JAK inhibitor-1 (Calbiochem) at varying concentrations for 72 hours, compared to the same cell line exposed to vehicle. Error bars indicate one standard deviation.

### DETAILED DESCRIPTION

By random sequencing of a dendritic cell (DC) complementary DNA (cDNA) library, Zhang et al. isolated a cDNA encoding CRLF2, which they termed CRL2. Zhang Wet al. (2001) Biochem Biophys Res Commun 281:878-83. Sequence analysis predicted that the 371-amino acid type I transmembrane protein contains an N-terminal signal peptide, four potential N-linked glycosylation sites, and two of four conserved cysteine residues. The protein also has a transmembrane domain and a 119-amino acid intracellular domain with a conserved membrane-proximal “box 1” motif and a potential signal-transducing tyrosine residue. Northern blot analysis revealed expression of a 4.5-kb transcript in spleen, peripheral blood leukocytes, and promyelocytic leukemia cells but not in other tissues or cell lines tested. Reverse transcriptase-polymerase chain reaction (RT-PCR) analysis detected expression in peripheral monocytes, monocyte-derived DCs, and other monocytic cell lines. Expression was upregulated in activated monocytes but not in T cells. Western blot analysis showed expression of a 48-kD FLAG-tagged protein, which is larger than the predicted molecular mass, suggesting that CRLF2 is indeed glycosylated.

Tonozuka et al. independently cloned CRLF2 cDNA from a human T lymphocyte cDNA library. Tonozuka Y et al. (2001) Cytogenet Cell Genet. 93:23-5. They found that the CRLF2 protein contains 2 fibronectin type III-like domains in the N-terminal extracellular region and box-1- and box-2-like motifs in the C-terminal intracellular region. CRLF2 shares 35% amino acid sequence identity with delta-1/TSLPR. Northern blot analysis detected CRLF2 transcripts of 1.6 kb and 0.9 kb in heart, skeletal muscle, kidney, and liver, as well as transcripts of 2.2 kb and 1.6 kb in fetal liver and of 0.9 kb in placenta and bone marrow.

By fluorescence in situ hybridization (FISH), Tonozuka et al. (2001) also mapped the human CRLF2 gene to the pseudoautosomal region, Xp22.3 and Yp11.3.

Reche et al. cloned CRLF2, which they termed TSLPR, as well as its ligand, TSLP. Reche P A et al. (2001) J Immunol 167:336-43. They noted that there is a soluble splice variant of mouse TSLPR and suggested that an analogous human molecule could act as a TSLP inhibitor.

Reche et al. (2001) also showed that expression of TSLPR and interleukin-7 receptor, together but not alone, induced a proliferative response to TSLP, but not to IL-7, indicating that the functional TSLP receptor consists of these two subunits. PCR analysis of cDNA libraries suggested that DCs and monocytes coexpress IL-7R and TSLPR. Incubation of DCs or monocytes with TSLP enhanced expression of CCL17, CCL18, CCL19, and CCL22. IL-7, on the other hand, induced expression of CCL17, CCL19, and CCL22, but also CXCL1, CXCL2, CXCL3, CXCL5, CXCL7, and CXCL8. Functional analysis indicated that TSLP enhances the DC maturation process, as evidenced by upregulation of DC markers and costimulatory molecules and stronger T-cell proliferation.

Using a mouse model of allergic skin inflammation elicited by repeated epicutaneous (EC) sensitization with ovalbumin (OVA) to tape-stripped skin, which mimics the scratching-inflicted injury associated with atopic dermatitis, He et al. found that TSLPR −/− mice had reduced inflammation, with fewer eosinophils and local Th2 cytokine expression, but unchanged splenocyte secretion of these cytokines. He R et al. (2008) Proc Natl Acad Sci USA 105:11875-80. Addition of TSLP significantly enhanced Th2 cytokine secretion in vitro by targeting TSLPR on antigen-specific T cells. Intradermal injection of anti-TSLP blocked the development of allergic skin inflammation after EC antigen challenge of OVA-immunized wild-type mice. He et al. (2008) proposed that TSLP is essential for antigen-driven Th2 cytokine secretion by skin-infiltrating effector T cells.

An amino acid sequence for full-length wild-type human CRLF2 is available as GenBank Accession No. NP_{—}071431. This sequence is incorporated herein as SEQ ID NO:1. Notably, amino acid residue 232 in SEQ ID NO:1 is Phe (phenylalanine; F).

As used herein, a F232C mutant of human CRLF2 polypeptide has Cys (cysteine; C) as amino acid residue 232 rather than Phe as provided in SEQ ID NO:1. In one embodiment, a F232C mutant of human CRLF2 polypeptide has an amino acid sequence at least 99% identical to SEQ ID NO:1 and includes a F232C mutation. An amino acid sequence at least 99% identical to SEQ ID NO:1 has at least 367 amino acid residues identical to SEQ ID NO:1. Thus in various embodiments, a F232C mutant of human CRLF2 polypeptide has 367, 368, 369, or 370 amino acid residues identical to SEQ ID NO:1 and includes a F232C mutation. In one embodiment a F232C mutant of human CRLF2 polypeptide is identical to SEQ ID NO:1 save for a F232C mutation. An amino acid sequence for a F232C mutant of human CRLF2 polypeptide that is identical to SEQ ID NO:1 save for a F232C mutation is provided as SEQ ID NO:3.

The CRLF2 Phe232 residue is near the junction of the extracellular and transmembrane domains. Mutations that introduce cysteine residues in this region of other receptor tyrosine kinases, such as RET, can activate signal transduction through intermolecular disulfide-bonded dimers. As described further herein, immunoblots in BaF3 cells expressing wild-type CRLF2 or CRLF2 F232C under both reducing and non-reducing conditions confirmed that CRLF2 F232C promotes constitutive dimerization. Under non-reducing conditions, the molecular weight of the CRLF2 F232C band, but not the wild-type band, was doubled, consistent with constitutive dimerization through the cysteine residues.

Except as may be expressly stated or otherwise evident from context, “CRLF2 F232C” as used herein refers to a mutant form of human CRLF2, the polypeptide of which has an amino acid sequence that is identical to SEQ ID NO:1 save for a F232C mutation. In one embodiment “CRLF2 F232C” refers to a homodimeric protein, each polypeptide of which has an amino acid sequence that is identical to SEQ ID NO:1 save for a F232C mutation.

Similar to CRLF2 F232C, it is believed that any F232C mutant of human CRLF2 polypeptide having an amino acid sequence at least 99% identical to SEQ ID NO:1 and including a F232C mutation similarly forms a homodimer under physiologic conditions. In one embodiment a F232C mutant of human CRLF2 polypeptide having an amino acid sequence at least 99% identical to SEQ ID NO:1 and including a F232C mutation forms a homodimer under physiologic conditions. Such homodimer formation can be assessed using any suitable method, including, for example, performing immunoblots in BaF3 cells expressing wild-type or mutant CRLF2 under both reducing and non-reducing conditions, where observation that the molecular weight of the mutant CRLF2 band, but not the wild-type band, doubles under non-reducing conditions, is consistent with constitutive dimerization through the cysteine residues, such as is described in the Examples below.

CRLF2 F232C is disclosed herein to have STAT5-activating activity. Similar to CRLF2 F232C, it is believed that any F232C mutant of human CRLF2 polypeptide having an amino acid sequence at least 99% identical to SEQ ID NO:1 and including a F232C mutation activates STAT5 signaling. In one embodiment a F232C mutant of human CRLF2 polypeptide having an amino acid sequence at least 99% identical to SEQ ID NO:1 and including a F232C mutation activates STAT5 signaling. Such activity can be assessed using any suitable method, including, for example, expression of the F232C mutant of human CRLF2 in cytokine-dependent BaF3 cells grown in the absence of interleukin 3 (IL-3), such as is described in the Examples below.

CRLF2 F232C is disclosed herein to be a gain-of-function mutation that has constitutive STAT5-activating activity. Similar to CRLF2 F232C, it is believed that any F232C mutant of human CRLF2 polypeptide having an amino acid sequence at least 99% identical to SEQ ID NO:1 and including a F232C mutation similarly has constitutive STAT5-activating activity. In one embodiment a F232C mutant of human CRLF2 polypeptide having an amino acid sequence at least 99% identical to SEQ ID NO:1 and including a F232C mutation has constitutive STAT5-activating activity. Such activity can be assessed using any suitable method, including, for example, expression of the F232C mutant of human CRLF2 in cytokine-dependent BaF3 cells grown in the absence of interleukin 3 (IL-3), such as is described in the Examples below.

Polypeptides of the invention may include one or more conservative or non-conservative amino acid substitutions, one or more amino acid additions, and/or one or more amino acid deletions as compared to SEQ ID NO:1. As used herein, a “conservative amino acid substitution” or “conservative substitution” refers to an amino acid substitution in which the substituted amino acid residue is of similar charge as the replaced residue and is of similar or smaller size than the replaced residue. Conservative substitutions of amino acids include substitutions made amongst amino acids within the following groups: (a) the small non-polar amino acids, A, M, I, L, and V; (b) the small polar amino acids, G, S, T and C; (c) the amido amino acids, Q and N; (d) the aromatic amino acids, F, Y and W; (e) the basic amino acids, K, R and H; and (f) the acidic amino acids, E and D. Substitutions which are charge neutral and which replace a residue with a smaller residue may also be considered “conservative substitutions” even if the residues are in different groups (e.g., replacement of phenylalanine with the smaller isoleucine). The term “conservative amino acid substitution” also refers to the use of amino acid analogs or variants. The term “non-conservative amino acid substitution” refers to any amino acid substitution other than a conservative amino acid substitution.

Methods for making amino acid substitutions, additions or deletions are well known in the art. The terms “conservative substitution”, “non-conservative substitutions”, “non-polar amino acids”, “polar amino acids”, and “acidic amino acids” are all used consistently with the prior art terminology. Each of these terms is well-known in the art and has been extensively described in numerous publications, including standard biochemistry textbooks, such as Biochemistry by Geoffrey Zubay, Addison-Wesley Publishing Co., 1986 edition, which describes conservative and non-conservative substitutions and properties of amino acids which lead to their definition as polar, non-polar or acidic.

In one embodiment a F232C mutant of human CRLF2 polypeptide having an amino acid sequence at least 99% identical to SEQ ID NO:1 and includes a F232C mutation further includes a heterologous polypeptide, such as a poly-histidine tag, an enzymatic marker, e.g., green fluorescent protein (GFP), or an immunoglobulin Fc gamma polypeptide or immunoglobulin constant heavy chain (C_{H}) region polypeptide. Such chimeric polypeptides can be prepared as fusion proteins using standard molecular biological methods.

An aspect of the invention is an isolated mutant human cytokine receptor-like factor 2 (CRLF2) polypeptide having an amino acid sequence at least 99% identical to SEQ ID NO:1 and comprising a F232C mutation. As used herein, an “isolated” polypeptide refers to a polypeptide that is substantially pure and free of other substances with which the polypeptide is ordinarily found in nature or in vivo systems to an extent practical and appropriate for its intended use. Similarly, as used herein, an “isolated” antibody refers to an antibody that is substantially pure and free of other substances with which the antibody is ordinarily found in nature or in vivo systems to an extent practical and appropriate for its intended use. Likewise, as used herein, an “isolated” nucleic acid refers to a nucleic acid molecule (i.e., polynucleotide) that is substantially pure and free of other substances with which the nucleic acid molecule is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polysaccharide. Because an isolated molecular species of the invention may be admixed with a pharmaceutically-acceptable carrier in a pharmaceutical preparation or be mixed with some of the components with which it is associated in nature, the molecular species may comprise only a small percentage by weight of the preparation. The molecular species is nonetheless substantially pure in that it has been substantially separated from the substances with which it may be associated in living systems.

The polypeptides and proteins of the invention can be used to generate antibodies, including monoclonal antibodies, that bind specifically to said polypeptides and proteins. For example, a polypeptide or protein of the invention can be injected into a mammal by any route of administration, e.g., subcutaneous, intramuscular, intraperitoneal, or intravenous, that is suitable for the purpose of immunizing the mammal against the polypeptide or protein. The polypeptide or protein can be administered to the mammal on one or more occasions, preferably on two or more occasions, with or without an adjuvant. Adjuvants include but are not limited to complete Freund's adjuvant, incomplete Freund's adjuvant, alum, aluminum phosphate, aluminum hydroxide, squalene, QS21, and CpG DNA. Monoclonal antibodies can be developed using standard methods first developed by Kohler and Milstein (1975) Nature 256:495-7.

In addition to polypeptides and proteins of the invention, the invention also includes nucleic acid molecules related to said polypeptides. An aspect of the invention is an isolated nucleic acid molecule that includes a sequence that encodes the mutant human cytokine receptor-like factor 2 (CRLF2) polypeptide of the invention. In one embodiment such nucleic acid molecule includes a sequence that encodes an amino acid sequence at least 99% identical to SEQ ID NO:1 and includes a F232C mutation. In various embodiments, a nucleic acid molecule of the invention encodes a polypeptide having 367, 368, 369, or 370 amino acid residues identical to SEQ ID NO:1 and includes a F232C mutation. In one embodiment a nucleic acid molecule of the invention encodes a polypeptide that is identical to SEQ ID NO:1 save for a F232C mutation, i.e., CRLF2 F232C. In one embodiment the polynucleotide that is encoded by a nucleic acid molecule of the invention has STAT5-activating activity. In one embodiment the polynucleotide that is encoded by a nucleic acid molecule of the invention has constitutive STAT5-activating activity.

A cDNA nucleic acid sequence encoding a polypeptide having an amino acid sequence provided by SEQ ID NO:1 is available as GenBank Accession No. NM_{—}022148, incorporated herein as SEQ ID NO:2. Nucleotides 17-1132 of SEQ ID NO:2 code for SEQ ID NO:1. Knowing the genetic code, it is possible to generate any number of alternative nucleic acid sequences that also encode a polypeptide having an amino acid sequence provided by SEQ ID NO:1.

CRLF2 F232C as disclosed herein was found to arise from a single nucleotide somatic mutation, substitution of G for T at nucleotide 711 of SEQ ID NO:2. This mutation results in a change from codon TTT (encoding Phe) to codon TGT (encoding Cys). In one embodiment a nucleic acid molecule that includes a sequence that encodes the mutant human cytokine receptor-like factor 2 (CRLF2) polypeptide of the invention is identical to SEQ ID NO:2 except for substitution of G for T at nucleotide 711 of SEQ ID NO:2. In one embodiment a nucleic acid molecule that includes a sequence that encodes the mutant human cytokine receptor-like factor 2 (CRLF2) polypeptide of the invention is identical to nucleotides 17-1132 of SEQ ID NO:2 except for substitution of G for T at nucleotide 711 of SEQ ID NO:2. In one embodiment a nucleic acid molecule that includes a sequence that encodes the mutant human cytokine receptor-like factor 2 (CRLF2) polypeptide of the invention is provided as SEQ ID NO:4.

In one embodiment a nucleic acid molecule of the invention is (a) a nucleic acid molecule that hybridizes under stringent conditions to SEQ ID NO:2, is at least 99 percent identical to nucleotides 17-1132 of SEQ ID NO:2, and includes a substitution of G for T at nucleotide 711 of SEQ ID NO:2, or (b) a complement of (a). A nucleic acid molecule that is at least 99 percent identical to nucleotides 17-1132 of SEQ ID NO:2 and includes a substitution of G for T at nucleotide 711 of SEQ ID NO:2 includes at least 1105 nucleotides identical to nucleotides 17-1132 of SEQ ID NO:2. In various embodiments the nucleic acid molecule includes 1105, 1106, 1107, 1108, 1109, 1110, 1111, 1112, 1113, 1114, or 1115 nucleotides identical (or complementary) to nucleotides 17-1132 of SEQ ID NO:2. In one embodiment the nucleic acid molecule is identical (or complementary) to nucleotides 17-1132 of SEQ ID NO:2 except for substitution of G for T at nucleotide 711 of SEQ ID NO:2. In one embodiment the nucleic acid molecule has a sequence provided by SEQ ID NO:4; in one embodiment the nucleic acid molecule has a sequence provided the complement of SEQ ID NO:4.

The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Third Edition, Cold Spring Harbor Laboratory Press, Cold Spring Harbor, N.Y., 2000, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15M sodium chloride/0.015M sodium citrate, pH7; SDS is sodium dodecyl sulphate; and EDTA is ethylenediaminetetraacetic acid. Modification of the hybridization conditions (for example, increasing the hybridization temperature or decreasing salt concentration) may be used to increase specificity and decrease hybridization of the probe to sequences that are less than 100% similar.

Also included in the invention are vectors that include nucleic acid molecules of the invention. For example, in one embodiment the invention is a vector that includes a nucleic acid molecule having a sequence that is identical to SEQ ID NO:2 except for substitution of G for T at nucleotide 711 of SEQ ID NO:2. In one embodiment the vector includes a nucleic acid molecule having a sequence that is provided as SEQ ID NO:4. Vectors of the invention can be used to replicate, modify, or express (through transcription and translation) nucleic acid molecules of the invention.

In one embodiment, an expression vector comprising any of the mutant CRLF2 nucleic acid molecules of the invention (e.g., a nucleic acid molecule that includes a sequence that encodes a mutant human CRLF2 polypeptide of the invention that is identical to SEQ ID NO:2 except for substitution of G for T at nucleotide 711 of SEQ ID NO:2), preferably operably linked to a promoter, is provided. In a related aspect, host cells transformed or transfected with such expression vectors also are provided. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, cloning vectors, expression vectors, and virus genomes. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., β-galactosidase or alkaline phosphatase, and genes which visibly affect the phenotype of transformed or transfected cells, hosts, colonies or plaques, e.g., green fluorescent protein. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ (upstream) regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

It will also be recognized that the invention embraces the use of the mutant CRLF2 encoding nucleic acid molecules in expression vectors. Expression vectors containing all the necessary elements for expression are commercially available and known to those skilled in the art. See, e.g., Sambrook et al., Molecular Cloning: A Laboratory Manual, Third Edition, Cold Spring Harbor Laboratory Press, 2000. Cells are genetically engineered by the introduction into the cells of an expression vector encoding a mutant CRLF2 polypeptide, fragments, or variants thereof. The host cell may be of a wide variety of tissue types, including fibroblasts (e.g., HEK 293, available from various commercial suppliers including Invitrogen, Carlsbad, Calif.), oocytes (e.g., CHO, available from various commercial suppliers including Invitrogen), and lymphocytes, and may be primary cells and cell lines. Specific examples include dendritic cells, peripheral blood leukocytes, bone marrow stem cells and embryonic stem cells. The expression vectors require that the pertinent sequence, i.e., those nucleic acids described herein, be operably linked to a promoter.

Systems for mRNA expression in mammalian cells are those such as pcDNA (Invitrogen) that contain a selectable marker (which facilitates the selection of stably transfected cell lines) and contain the human cytomegalovirus (CMV) enhancer-promoter sequences. Gateway™ vectors such as pCMVSport-6 vectors from Invitrogen are particularly suitable for rapid cloning. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. See, for example, Nakayama et al. (2005) J Virol 79:8870-7. Another expression vector is the pEF-BOS plasmid containing the promoter of polypeptide Elongation Factor 1, which stimulates efficiently transcription in vitro. The plasmid is described by Mizushima and Nagata (1990) Nucleic Acids Res 18:5322, and its use in transfection experiments is disclosed by, for example, Demoulin (1996) Mol Cell Biol 16:4710-6. Still another expression vector is an adenovirus, described by Stratford-Perricaudet (1992), which is defective for E1 and E3 proteins (J Clin Invest 90:626-30). The use of the adenovirus as an Adeno.P1A recombinant is described by Warnier et al. (1996), in intradermal injection in mice for immunization against HA (Int J Cancer 67:303-10). Other examples include Ad5 based Adenoviral expression vectors such as those described in Catalucci et al. (2005) J Virol 79:6400-9.

According to further aspects of the invention, compositions containing the nucleic acid molecules, polypeptides and immunogenic fragments thereof, and binding agents of the invention are provided. The compositions contain any of the foregoing therapeutic agents in an optional pharmaceutically acceptable carrier. Thus, in a related aspect, the invention provides a method for forming a medicament that involves placing a therapeutically effective amount of the therapeutic agent in the pharmaceutically acceptable carrier to form one or more doses. The effectiveness of treatment or prevention methods of the invention can be determined using the diagnostic methods described herein.

The invention also includes binding agents, such as antibodies and antigen-binding fragments, that bind to mutant CRLF2 polypeptide or mutant CRLF2 protein. Such agents can be used in methods of the invention including methods for the diagnosis and/or treatment of B-ALL. Such agents also may be used to inhibit the native activity of the mutant CRLF2 polypeptides, for example, by binding to such polypeptides in vivo.

The binding agents of the invention bind to a mutant CRLF2 polypeptide, including immunogenic fragments thereof. In certain embodiments, the binding agent is an antibody or antibody fragment, for example, an Fab or F(ab′)₂ fragment of an antibody or a single chain variable domain (scFv) antibody fragment. Typically, the fragment includes a CDR3 region that is selective for a mutant CRLF2 antigen of the invention. Any of the various types of antibodies can be used for this purpose, including polyclonal antibodies, monoclonal antibodies, chimeric antibodies, humanized antibodies, fully human antibodies, and scFv antibody fragments.

Significantly, as is well-known in the art, only a small portion of an antibody molecule, the paratope, is involved in the binding of the antibody to its epitope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen-binding sites of an intact antibody. Similarly, an antibody from which the Fc region has been enzymatically cleaved, or which has been produced without the Fc region, designated a Fab fragment, retains one of the antigen-binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762, and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present disclosure also provides for F(ab′)₂, Fab, Fv, and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present disclosure also includes so-called single chain antibodies.

In a preferred embodiment, an anti-mutant CRLF2 antibody binds specifically to a mutant CRLF2 polypeptide or homodimeric protein. As used herein, an antibody “binds specifically” to a mutant CRLF2 when it binds the mutant CRLF2 with at least 10-fold greater affinity than for wild-type CRLF2. In one embodiment, an antibody binds specifically to a mutant CRLF2 when it binds the mutant CRLF2 with at least 20-fold greater affinity than for wild-type CRLF2. In one embodiment, an antibody binds specifically to a mutant CRLF2 when it binds the mutant CRLF2 with at least 50-fold greater affinity than for wild-type CRLF2. In one embodiment, an antibody binds specifically to a mutant CRLF2 when it binds the mutant CRLF2 with at least 100-fold greater affinity than for wild-type CRLF2.

In one embodiment, the antibody that binds specifically to the mutant CRLF2 binds the mutant CRLF2 with a K_{D} of at least 1×10⁻⁹ M. In one embodiment, the antibody binds the mutant CRLF2 with a K_{D} of at least 1×10⁻¹⁰ M. In one embodiment, the antibody binds the mutant CRLF2 with a K_{D} of at least 1×10⁻¹¹ M.

The antibody can bind to a linear determinant, for example an epitope that includes the F232C mutation (i.e., Cys 232). In one embodiment, the antibody binds to an epitope including the sequence PTPPKPKLSKClLISSLAILL (SEQ ID NO:5), wherein Cys 232 is part of the epitope. In one embodiment, the antibody binds to an epitope within the sequence PTPPKPKLSKClLISSLAILL (SEQ ID NO:5), wherein Cys 232 is part of the epitope.

Alternatively or in addition, the antibody can bind to a conformational (e.g., 3-dimensional) epitope that is characteristic of the homodimer. In such an embodiment, the antibody can but need not necessarily bind an epitope that includes Cys 232.

In one embodiment the antibody or antigen-binding fragment thereof is a multivalent, multispecific antibody or multivalent, multispecific antigen-binding fragment thereof. A multivalent, multispecific antibody is an engineered monoclonal antibody that recognizes at least two distinct antigens or epitopes. In one embodiment the antibody or antigen-binding fragment thereof is a bispecific antibody or bispecific antigen-binding fragment thereof. A bispecific antibody is an engineered monoclonal antibody that recognizes two distinct antigens or epitopes. Such antibody can have two distinct antigen-binding domains, each recognizing an antigen or epitope that is distinct from that recognized by the other antigen-binding domain. General techniques for the preparation of multivalent antibodies may be found, for example, in Nisonhoff et al. (1961) Arch Biochem Biophys 93:470 (1961), Hammerling et al. (1968) J Exp Med 128:1461, and U.S. Pat. No. 4,331,647. See also U.S. Pat. No. 6,458,933. Examples of bispecific antibodies known in the art include antibodies 2B1, 520C9xH22, mDX-H210, and MDX447. A bispecific antibody or bispecific antigen-binding fragment thereof according to the instant invention can bind wild-type or mutant CRLF2, on the one hand, and a B-cell antigen, such as CD10, CD19, or CD20, on the other.

CD10 is also known as common acute lymphocytic leukemia antigen (CALLA), a cell surface enzyme with neutral metalloendopeptidase activity which inactivates a variety of biologically active peptides. CD10 is expressed on the cells of lymphoblastic, Burkitt's, and follicular germinal center lymphomas, immature B cells within adult bone marrow, and on cells from patients with chronic myelocytic leukemia (CML). CD19 is a type-I transmembrane glycoprotein of 95 kDa that belongs to the immunoglobulin superfamily. CD19 is expressed on B cells throughout most stages of B-cell differentiation, although its expression is down-regulated during their terminal differentiation to plasma cells. Expression of CD19 is also found in the majority of B cell-derived malignancies. CD20 is a non-glycosylated phosphoprotein expressed on the surface of all mature B cells. In addition, CD20 is found on B-cell lymphomas, hairy cell leukemia, and B-cell chronic lymphocytic leukemia. It is also found on skin/melanoma cancer stem cells.

A number of monoclonal anti-CD20 antibodies are currently in clinical use. Rituxan®, an anti-CD20 antibody, has been approved for the treatment of patients with non-Hodgkin's lymphoma (NHL) who have failed initial therapy. Zevalin, which is essentially Rituxan® linked to Yttrium 90 (⁹⁰Y), is approved for treatment of patients with NHL who have failed initial chemotherapy. Recently the FDA also approved tositumomab (Bexxar®), which is an anti-CD20 antibody linked to iodine 131 (¹³¹I). Additional anti-CD20 antibodies currently under development include AME-133v (Applied Molecular Evolution), Ocrelizumab (Roche), Ofatumumab (Genmab), TRU-015 (Trubion/Wyeth), and IMMU-106 (Immunomedics).

The antibody or antigen-binding fragment thereof can be used as a targeting means for delivery of a therapeutic agent to cells expressing the mutant CRLF2. For example, the antibody can be conjugated to a toxin or toxic moiety. Toxins useful for this purpose can include, without limitation, at least an enzymatically active portion of diphtheria toxin (DT), pseudomonas exotoxin A (PEA), ricin A toxin, C. botulinum C2 toxin, and gelonin. Toxic moieties can include, without limitation, radionuclides such as ⁹⁰Y, ¹⁰⁵Rh, ¹³¹I, ¹⁵³SM, ¹⁸⁶Re, ¹⁸⁸Re, ¹⁹⁸Au, and ²¹¹At. See, for example, U.S. Pat. No. 4,837,003. In one embodiment the conjugate is a covalent conjugate. In one embodiment the conjugate is a recombinant fusion protein.

The invention also includes nucleic acid molecules that bind specifically to nucleic acid molecules encoding the mutant CRLF2 and reduce expression of the mutant CRLF2. These nucleic acid molecules include antisense and RNA interference (RNAi).

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Antisense oligonucleotides that selectively bind to a nucleic acid molecule encoding a mutant CRLF2 as disclosed herein are particularly preferred. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. Based upon the nucleotide sequences of nucleic acid molecules encoding wild-type CRLF2 or mutant CRLF2 F232C, (e.g., SEQ ID NOs. 2 and 4) or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least about 10 and, more preferably, at least about 15 consecutive nucleotides which are complementary to the target sequence, although in certain cases modified oligonucleotides as short as 7 bases in length have been used successfully as antisense oligonucleotides. See Wagner et al. (1995) Nat Med 1:1116-8. Most preferably, the antisense oligonucleotides comprise a complementary sequence of 20-30 nucleotides. Although oligonucleotides may be chosen which are antisense to any region of the gene or mRNA transcripts, in preferred embodiments the antisense oligonucleotides may generally correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al. (1994) Cell Mol Neurobiol 14:439-57) and at which proteins, e.g., transcription factors, are not expected to bind. In one embodiment the antisense is targeted to a site that includes sequence encoding the mutant Cys 232.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art-recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters, and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 2′ position and other than a phosphate group at the 5′ position. Thus modified oligonucleotides may include a 2′-O-alkylated ribose group. In addition, modified oligonucleotides may include sugars such as arabinose instead of ribose.

The present invention, thus, contemplates pharmaceutical preparations containing modified antisense molecules that are complementary to and hybridizable with, under physiological conditions, nucleic acid molecules encoding a mutant CRLF2 polypeptide, together with pharmaceutically acceptable carriers. Antisense oligonucleotides may be administered as part of a pharmaceutical composition. In this latter embodiment, it may be preferable that a slow intravenous administration be used. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a subject.

The methods of the invention also encompass use of isolated short RNA that directs the sequence-specific degradation of a mutant CRLF2 mRNA through a process known as RNA interference (RNAi). The process is known to occur in a wide variety of organisms, including embryos of mammals and other vertebrates. It has been demonstrated that double-stranded RNA (dsRNA) is processed to RNA segments 21-23 nucleotides (nt) in length, and furthermore, that they mediate RNA interference in the absence of longer dsRNA. Thus, these 21-23 nt fragments are sequence-specific mediators of RNA degradation and are referred to herein as short interfering RNA (siRNA) or RNAi. Methods of the invention encompass the use of these fragments (or recombinantly produced or chemically synthesized oligonucleotides of the same or similar nature) to enable the targeting of mutant CRLF2 mRNAs for degradation in mammalian cells useful in the therapeutic applications discussed herein.

The methods for design of the RNA's that mediate RNAi and the methods for transfection of the RNAs into cells and animals is well known in the art and are readily commercially available. Verma et al. (2004) J Clin Pharm Ther 28(5):395-404; Mello et al. (2004) Nature 431 (7006):338-42; Dykxhoorn et al. (2003) Nat Rev Mol Cell Biol 4(6):457-67; Proligo (Hamburg, Germany); Dharmacon Research (Lafayette, Colo., USA); Pierce Chemical (part of Perbio Science, Rockford, Ill., USA), Glen Research (Sterling, Va., USA), ChemGenes (Ashland, Mass., USA), and Cruachem (Glasgow, UK). The RNAs are preferably chemically synthesized using appropriately protected ribonucleoside phosphoramidites and a conventional DNA/RNA synthesizer. Most conveniently, siRNAs are obtained from commercial RNA oligonucleotide synthesis suppliers. In general, RNAs are not difficult to synthesize and are readily provided in a quality suitable for RNAi. A typical 0.2 mmol-scale RNA synthesis provides about 1 milligram of RNA, which is sufficient for 1000 transfection experiments using a 24-well tissue culture plate format.

The mutant CRLF2 cDNA-specific siRNA is designed preferably by selecting a sequence that is not within 50-100 bp of the start codon and the termination codon, avoids intron regions, avoids stretches of 4 or more bases such as AAAA, CCCC, avoids regions with GC content <30% or >60%, avoids repeats and low complexity sequence, and avoids single nucleotide polymorphism sites. The mutant CRLF2 siRNA may be designed by a search for a 23-nt sequence motif AA(N19), where A is adenine and N is any nucleobase. If no suitable sequence is found, then a 23-nt sequence motif NA(N21) may be used with conversion of the 3′ end of the sense siRNA to TT, where T is thymine. Alternatively, the mutant CRLF2 siRNA can be designed by a search for NAR(N17)YNN, where R is purine and Y is pyrimidine. The target sequence may have a GC content of around 50%. The siRNA targeted sequence may be further evaluated using a BLAST homology search to avoid off-target effects on other genes or sequences. Negative controls are designed by scrambling targeted siRNA sequences. The control RNA preferably has the same length and nucleotide composition as the siRNA but has at least 4-5 bases mismatched to the siRNA. The RNA molecules of the present invention can comprise a 3′ hydroxyl group. The RNA molecules can be single-stranded or double-stranded; such molecules can be blunt-ended or comprise overhanging ends (e.g., 5′, 3′) from about 1 to about 6 nucleotides in length (e.g., pyrimidine nucleotides, purine nucleotides). In order to further enhance the stability of the RNA of the present invention, the 3′ overhangs can be stabilized against degradation. The RNA can be stabilized by including purine nucleotides, such as adenine or guanine nucleotides. Alternatively, substitution of pyrimidine nucleotides by modified analogues, e.g., substitution of uridine 2-nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

The RNA molecules used in the methods of the present invention can be obtained using a number of techniques known to those of skill in the art. For example, the RNA can be chemically synthesized or recombinantly produced using methods known in the art. Such methods are described in U.S. Published Patent Application Nos. US2002-0086356A1 and US2003-0206884A1 that are hereby incorporated by reference in their entirety.

The methods described herein are used to identify or obtain RNA molecules that are useful as sequence-specific mediators of mutant CRLF2 mRNA degradation and, thus, for inhibiting mutant CRLF2 activity. Expression of mutant CRLF2 can be inhibited in humans in order to prevent the protein from being translated and thus contributing to the uncontrolled proliferation of malignant precursor B cells.

The RNA molecules may also be isolated using a number of techniques known to those of skill in the art. For example, gel electrophoresis can be used to separate RNAs from the combination, gel slices comprising the RNA sequences removed and RNAs eluted from the gel slices. Alternatively, non-denaturing methods, such as non-denaturing column chromatography, can be used to isolate the RNA produced. In addition, chromatography (e.g., size exclusion chromatography), glycerol gradient centrifugation, affinity purification with antibody can be used to isolate RNAs.

Any RNA can be used in the methods of the present invention, provided that it has sufficient homology to the mutant CRLF2 gene to mediate RNAi. The RNA for use in the present invention can correspond to the entire mutant CRLF2 gene or a portion thereof. There is no upper limit on the length of the RNA that can be used. For example, the RNA can range from about 21 base pairs (bp) of the gene to the full length of the gene or more. In one embodiment, the RNA used in the methods of the present invention is about 1000 bp in length. In another embodiment, the RNA is about 500 bp in length. In yet another embodiment, the RNA is about 22 bp in length. In certain embodiments the preferred length of the RNA of the invention is 21 to 23 nucleotides.

In various certain embodiments, the antisense or RNAi has a sequence that is identical to, corresponds to, or is complementary to one of the following sequences:

(SEQ ID NO: 6)
atgcctgtgcagagacaccaacgcctcccaaaccaaagctgtccaaa
tgtattttaatttccagcctggccatccttctgatggtgtctctcct
ccttctgtctttatggaaattatggag
(Exon 6 (663-783) of GenBank Accession No.
NM_022148 (human CRLF2) with 711t > g))

| | SEQ ID NO: |
|---|---|
| 23-mers: | |
| cccaaaccaaagctgtccaaatg | 7 |
| ccaaaccaaagctgtccaaatgt | 8 |
| caaaccaaagctgtccaaatgta | 9 |
| aaaccaaagctgtccaaatgtat | 10 |
| aaccaaagctgtccaaatgtatt | 11 |
| accaaagctgtccaaatgtattt | 12 |
| ccaaagctgtccaaatgtatttt | 13 |
| caaagctgtccaaatgtatttta | 14 |
| aaagctgtccaaatgtattttaa | 15 |
| aagctgtccaaatgtattttaat | 16 |
| agctgtccaaatgtattttaatt | 17 |
| gctgtccaaatgtattttaattt | 18 |
| ctgtccaaatgtattttaatttc | 19 |
| tgtccaaatgtattttaatttcc | 20 |
| gtccaaatgtattttaatttcca | 21 |
| tccaaatgtattttaatttccag | 22 |
| ccaaatgtattttaatttccagc | 23 |
| caaatgtattttaatttccagcc | 24 |
| aaatgtattttaatttccagcct | 25 |
| aatgtattttaatttccagcctg | 26 |
| atgtattttaatttccagcctgg | 27 |
| tgtattttaatttccagcctggc | 28 |
| gtattttaatttccagcctggcc | 29 |
| 22-mers: | |
| ccaaaccaaagctgtccaaatg | 30 |
| caaaccaaagctgtccaaatgt | 31 |
| aaaccaaagctgtccaaatgta | 32 |
| aaccaaagctgtccaaatgtat | 33 |
| accaaagctgtccaaatgtatt | 34 |
| ccaaagctgtccaaatgtattt | 35 |
| caaagctgtccaaatgtatttt | 36 |
| aaagctgtccaaatgtatttta | 37 |
| aagctgtccaaatgtattttaa | 38 |
| agctgtccaaatgtattttaat | 39 |
| gctgtccaaatgtattttaatt | 40 |
| ctgtccaaatgtattttaattt | 41 |
| tgtccaaatgtattttaatttc | 42 |
| gtccaaatgtattttaatttcc | 43 |
| tccaaatgtattttaatttcca | 44 |
| ccaaatgtattttaatttccag | 45 |
| caaatgtattttaatttccagc | 46 |
| aaatgtattttaatttccagcc | 47 |
| aatgtattttaatttccagcct | 48 |
| atgtattttaatttccagcctg | 49 |
| tgtattttaatttccagcctgg | 50 |
| gtattttaatttccagcctggc | 51 |
| 21-mers: | |
| caaaccaaagctgtccaaatg | 52 |
| aaaccaaagctgtccaaatgt | 53 |
| aaccaaagctgtccaaatgta | 54 |
| accaaagctgtccaaatgtat | 55 |
| ccaaagctgtccaaatgtatt | 56 |
| caaagctgtccaaatgtattt | 57 |
| aaagctgtccaaatgtatttt | 58 |
| aagctgtccaaatgtatttta | 59 |
| agctgtccaaatgtattttaa | 60 |
| gctgtccaaatgtattttaat | 61 |
| ctgtccaaatgtattttaatt | 62 |
| tgtccaaatgtattttaattt | 63 |
| gtccaaatgtattttaatttc | 64 |
| tccaaatgtattttaatttcc | 65 |
| ccaaatgtattttaatttcca | 66 |
| caaatgtattttaatttccag | 67 |
| aaatgtattttaatttccagc | 68 |
| aatgtattttaatttccagcc | 69 |
| atgtattttaatttccagcct | 70 |
| tgtattttaatttccagcctg | 71 |
| gtattttaatttccagcctgg | 72 |
| 20-mers: | |
| aaaccaaagctgtccaaatg | 73 |
| aaccaaagctgtccaaatgt | 74 |
| accaaagctgtccaaatgta | 75 |
| ccaaagctgtccaaatgtat | 76 |
| caaagctgtccaaatgtatt | 77 |
| aaagctgtccaaatgtattt | 78 |
| aagctgtccaaatgtatttt | 79 |
| agctgtccaaatgtatttta | 80 |
| gctgtccaaatgtattttaa | 81 |
| ctgtccaaatgtattttaat | 82 |
| tgtccaaatgtattttaatt | 83 |
| gtccaaatgtattttaattt | 84 |
| tccaaatgtattttaatttc | 85 |
| ccaaatgtattttaatttcc | 86 |
| caaatgtattttaatttcca | 87 |
| aaatgtattttaatttccag | 88 |
| aatgtattttaatttccagc | 89 |
| atgtattttaatttccagcc | 90 |
| tgtattttaatttccagcct | 91 |
| gtattttaatttccagcctg | 92 |
| 19-mers: | |
| aaccaaagctgtccaaatg | 93 |
| accaaagctgtccaaatgt | 94 |
| ccaaagctgtccaaatgta | 95 |
| caaagctgtccaaatgtat | 96 |
| aaagctgtccaaatgtatt | 97 |
| aagctgtccaaatgtattt | 98 |
| agctgtccaaatgtatttt | 99 |
| gctgtccaaatgtatttta | 100 |
| ctgtccaaatgtattttaa | 101 |
| tgtccaaatgtattttaat | 102 |
| gtccaaatgtattttaatt | 103 |
| tccaaatgtattttaattt | 104 |
| ccaaatgtattttaatttc | 105 |
| caaatgtattttaatttcc | 106 |
| aaatgtattttaatttcca | 107 |
| aatgtattttaatttccag | 108 |
| atgtattttaatttccagc | 109 |
| tgtattttaatttccagcc | 110 |
| gtattttaatttccagcct | 111 |
| 18-mers: | |
| accaaagctgtccaaatg | 112 |
| ccaaagctgtccaaatgt | 113 |
| caaagctgtccaaatgta | 114 |
| aaagctgtccaaatgtat | 115 |
| aagctgtccaaatgtatt | 116 |
| agctgtccaaatgtattt | 117 |
| gctgtccaaatgtatttt | 118 |
| ctgtccaaatgtatttta | 119 |
| tgtccaaatgtattttaa | 120 |
| gtccaaatgtattttaat | 121 |
| tccaaatgtattttaatt | 123 |
| ccaaatgtattttaattt | 124 |
| caaatgtattttaatttc | 125 |
| aaatgtattttaatttcc | 126 |
| aatgtattttaatttcca | 127 |
| atgtattttaatttccag | 128 |
| tgtattttaatttccagc | 129 |
| gtattttaatttccagcc | 130 |
| 17-mers: | |
| ccaaagctgtccaaatg | 131 |
| caaagctgtccaaatgt | 132 |
| aaagctgtccaaatgta | 133 |
| aagctgtccaaatgtat | 134 |
| agctgtccaaatgtatt | 135 |
| gctgtccaaatgtattt | 136 |
| ctgtccaaatgtatttt | 137 |
| tgtccaaatgtatttta | 138 |
| gtccaaatgtattttaa | 139 |
| tccaaatgtattttaat | 140 |
| ccaaatgtattttaatt | 141 |
| caaatgtattttaattt | 142 |
| aaatgtattttaatttc | 143 |
| aatgtattttaatttcc | 144 |
| atgtattttaatttcca | 145 |
| tgtattttaatttccag | 146 |
| gtattttaatttccagc | 147 |
| 16-mers: | |
| caaagctgtccaaatg | 148 |
| aaagctgtccaaatgt | 149 |
| aagctgtccaaatgta | 150 |
| agctgtccaaatgtat | 151 |
| gctgtccaaatgtatt | 152 |
| ctgtccaaatgtattt | 153 |
| tgtccaaatgtatttt | 154 |
| gtccaaatgtatttta | 155 |
| tccaaatgtattttaa | 156 |
| ccaaatgtattttaat | 157 |
| caaatgtattttaatt | 158 |
| aaatgtattttaattt | 159 |
| aatgtattttaatttc | 160 |
| atgtattttaatttcc | 161 |
| tgtattttaatttcca | 162 |
| gtattttaatttccag | 163 |
| 15-mers: | |
| aaagctgtccaaatg | 164 |
| aagctgtccaaatgt | 165 |
| agctgtccaaatgta | 166 |
| gctgtccaaatgtat | 167 |
| ctgtccaaatgtatt | 168 |
| tgtccaaatgtattt | 169 |
| gtccaaatgtatttt | 170 |
| tccaaatgtatttta | 171 |
| ccaaatgtattttaa | 172 |
| caaatgtattttaat | 173 |
| aaatgtattttaatt | 174 |
| aatgtattttaattt | 175 |
| atgtattttaatttc | 176 |
| tgtattttaatttcc | 178 |
| gtattttaatttcca | 179 |

The invention further provides a method of treating precursor B-cell acute lymphoblastic leukemia (B-ALL). In one embodiment the method includes administering to a subject having B-ALL, wherein the B-ALL is characterized by mutant human cytokine receptor-like factor 2 (CRLF2) polypeptide having an amino acid sequence at least 99% identical to SEQ ID NO:1 and comprising a F232C mutation, an effective amount of an agent that inhibits signaling by the mutant CRLF2 to treat the B-ALL. As used herein, an “agent that inhibits signaling by the mutant CRLF2” is any agent that reduces the expression or the activity of mutant CRLF2. Also as used herein, “signaling by the mutant CRLF2” refers to any signal originating from and downstream of CRLF2, including, for example, activation of JAK2, STAT5, or ERK by the mutant CRLF2.

In one embodiment, an agent that inhibits signaling by the mutant CRLF2 is an anti-mutant CRLF2 antibody or antigen-binding fragment thereof, as disclosed herein. The antibody or antigen-binding fragment thereof can optionally be a multivalent, multispecific antibody or multivalent, multispecific antigen-binding fragment thereof, as described above. The antibody or antigen-binding fragment thereof can optionally be conjugated with a toxin or toxic moiety, as described above.

In one embodiment, an agent that inhibits signaling by the mutant CRLF2 is an antisense to the mutant CRLF2, as disclosed herein.

In one embodiment, an agent that inhibits signaling by the mutant CRLF2 is an RNAi, as disclosed herein.

In one embodiment, an agent that inhibits signaling by the mutant CRLF2 is a JAK inhibitor, including, for example, a JAK2 inhibitor. JAK2 abnormalities have previously been associated with hematologic malignancies and other hematologic conditions other than B-ALL. For example, JAK2 V617F mutation has been reported only in myeloid neoplasms, with a high frequency in polycythemia vera, essential thrombocythemia, primary myelofibrosis, and refractory anemia with ringed sideroblast and thrombocytosis. JAK mutations were recently reported in children with Down Syndrome and B-ALL. Bercovich et al. (2008) Lancet 372:1484-92; Kearney et al. (2009) Blood 113:646-8. A number of specific JAK2 inhibitors, including INCB018424 (Incyte, Wilmington, Del.), TG101209 (TargeGen, San Diego, Calif.), TG101348 (TargeGen), XL019 (Excelixis, So. San Francisco, Calif.), and TG10134841 (TargeGen) are currently under development and in clinical trials for the treatment of various myeloproliferative neoplasms (which do not include B-ALL). A number of non-specific JAK2 inhibitors are also currently in clinical trials. These include, for example, CEP-701 (an FLT3 inhibitor, Lestaurtinib, Cephalon, West Chester, Pa.), tipifarnib (a farnesyltransferase inhibitor, Zarnestra, Johnson & Johnson, Raritan, N.J.), ITF2357 (an HDAC inhibitor, Italfarmaco, Cinisello Balsamo, Italy), and hypomethylating agents. See, for example, Alabdulaali (2009) Hematology Reviews 1:e10.

In one embodiment, an agent that inhibits signaling by the mutant CRLF2 is a protein kinase C (PKC) inhibitor, including, for example, PKC142, Go6976, UCN-01, PKC412 and k252a.

In one embodiment, an agent that inhibits signaling by the mutant CRLF2 is a heat shock protein 90 (HSP90) inhibitor, including, for example, AUY 922 (Novartis).

Of course, any combination of the foregoing inhibitors or types of inhibitors of signaling by the mutant CRLF2 is embraced by the invention. In addition, any one or combination of the foregoing inhibitors may be combined with any other suitable method for treating B-ALL, including, for example, chemotherapy, radiation therapy, bone marrow transplant or allogeneic stem cell transplant, and combinations thereof. Some of the drugs used to treat ALL are clofarabine, cytarabine, daunorubicin, methotrexate, mitoxantrone, cyclophosphamide, vincristine, pegaspargase, imatinib mesylate, prednisone, and dexamethasone.

As used herein, to “treat” or “treating” a disease or condition refers to reducing or alleviating, at least to a significant extent, at least one objective manifestation, e.g., symptom or sign, of the disease or condition. For example, in one embodiment treating may result in a partial remission of B-ALL, and in one embodiment treating may result in a complete remission of B-ALL.

The invention further provides a method for identifying a subject at increased risk of mortality from precursor B-cell acute lymphoblastic leukemia (B-ALL). In one embodiment the method includes the steps of performing an assay on a sample isolated from a subject, wherein the assay detects presence of mutant human cytokine receptor-like factor 2 (CRLF2) characterized by an amino acid mutation F232C, and identifying the subject as having increased risk of mortality from B-ALL when the performing the assay detects the presence of the mutant CRLF2 in the sample. In one embodiment, the mutant human CRLF2 comprises a polypeptide having an amino acid sequence that is at least 99 percent identical to SEQ ID NO:1 and includes a F232C mutation, as described above.

In one embodiment the method includes the step of determining the presence of mutant human CRLF2 characterized by an amino acid mutation F232C by performing an assay on a sample isolated from a subject, wherein the presence of the mutant human CRLF2 in the sample indicates the subject is at increased risk of mortality from B-ALL.

In one embodiment the method includes the step of detecting the presence of mutant human CRLF2 characterized by an amino acid mutation F232C in an assay performed on a sample isolated from a subject, wherein the presence of the mutant human CRLF2 in the sample indicates the subject is at increased risk of mortality from B-ALL.

As used herein, a “subject” refers to a human.

In one embodiment, the subject has B-ALL. In such embodiment, the subject has been diagnosed as having B-ALL. In one embodiment, the subject is suspected of having B-ALL. In such embodiment, the subject has not been diagnosed as having B-ALL.

In one embodiment the subject has started or received treatment for B-ALL; in another embodiment the subject has not started or received treatment for B-ALL. In one embodiment, the subject is currently receiving treatment for B-ALL. In one embodiment, the subject has completed a course of treatment for B-ALL.

In one embodiment the subject has completed a course of treatment for B-ALL and the method is used to probe for the presence of residual disease when the subject is in apparent complete remission. In one embodiment the subject has completed a course of treatment for B-ALL that is characterized by a mutant CRLF2 characterized by an amino acid mutation F232C. In one embodiment the subject has completed a course of treatment for B-ALL that is characterized by CRLF2 F232C. For example, in one embodiment one or more specific PCR or RT-PCR primers that include or correspond to the 711t>g mutation are used to determine if the subject has residual molecularly detectable B-ALL. The method may be coupled to a method for treating residual disease when the method detects the presence of residual disease.

The assay can be any assay suitable for detecting the presence of mutant human cytokine receptor-like factor 2 (CRLF2) characterized by an amino acid mutation F232C. In one embodiment the assay is a mutation-specific assay. The assay in one embodiment is based on detecting a mutant CRLF2 protein. In one embodiment, the assay is based on detecting a mutant CRLF2 polypeptide. In one embodiment, the mutant human CRLF2 polypeptide has an amino acid sequence that is at least 99 percent identical to SEQ ID NO:1 and includes a F232C mutation, as described above. The protein or polypeptide assay may be based, for example, on the use of an antibody, or antigen-binding fragment thereof, that binds specifically to the protein or polypeptide. Such assay can be performed, for example, as an enzyme-linked immunosorbent assay (ELISA) or as a Western blot. General methods for performing ELISAs and Western blots are well known in the art. For this particular method, in one embodiment a first (primary or capture) antibody can be an anti-mutant CRLF2 antibody or antigen-binding fragment thereof, such as is disclosed herein, and a second (secondary, sandwich, or reporter) antibody can be a detectable antibody that binds specifically to the first antibody. Such second antibody may be linked to an enzyme such as horseradish peroxidase (HRP), or the second antibody may be linked to a chromogen or fluorochrome such as green fluorescent protein or other optically detectable marker. The foregoing examples of detectable antibodies are not meant to be limiting. In an alternative embodiment, an indirect ELISA may be used, whereby no primary or capture antibody is used, and sample is probed using a single, enzyme-linked or otherwise detectably-labeled antibody that binds specifically to the mutant CRLF2 protein or polypeptide.

The assay may conveniently be performed or adapted for use in an array, for example using a multiwell plate and a suitable multiwell plate reader device. Alternatively or in addition, the assay may be conveniently adapted for high-throughput screening, for example by using suitable multichannel pipetting devices and/or robotic devices designed for this purpose, examples of which are commercially available.

In one embodiment, the assay is a Western blot performed under reducing and non-reducing conditions so that mutant CRLF2 homodimer can be detected and distinguished from monomeric CRLF2. Since wild-type CRLF2 does not form homodimers, in this embodiment the antibody need not necessarily bind specifically to mutant CRLF2, provided that it does bind specifically to CRLF2. Such antibody can detect the presence of higher molecular weight species, corresponding to homodimer, as well as lower molecular weight species corresponding to monomer.

In one embodiment, the assay is based on detecting a nucleic acid molecule that encodes the mutant CRLF2 polypeptide. Without limitation, the assay in this embodiment can be based on standard methods that involve sequencing, amplifying, or hybridizing to a relevant target sequence in the nucleic acid molecule that encodes the mutant CRLF2 polypeptide. For example, in one embodiment cDNA for CRLF2 is prepared from a sample using standard reverse transcriptase-polymerase chain reaction RT-PCR with at least one oligonucleotide primer, e.g., a sense primer, that is specific for CRLF2; the cDNA is then sequenced and the obtained sequence is compared to corresponding sequence encoding wild-type CRLF2 or corresponding sequence encoding CRLF2 F232C.

As another example, in one embodiment a northern blot is performed using mRNA prepared from a sample and a hybridization probe that is complementary to a relevant target sequence in the nucleic acid molecule that encodes the mutant CRLF2 polypeptide. The hybridization can be carried out under highly stringent conditions whereby the probe binds to mRNA encoding mutant CRLF2 but not to mRNA encoding wild-type CRLF2. Probes for northern blotting are composed of nucleic acids with a complementary sequence to all or part of the RNA of interest. They can be DNA, RNA, or oligonucleotides with a minimum of 25 complementary bases to the target sequence. For example, a suitable DNA probe could be a DNA oligonucleotide having a sequence ctcccaaaccaaagctgtccaaatg (SEQ ID NO:180), agctgtccaaatgtattttaatttc (SEQ ID NO:181), or gtattttaatttccagcctggccat (SEQ ID NO:182). RNA probes (riboprobes) that are transcribed in vitro are able to withstand more rigorous washing steps, thereby reducing some of the background noise. Commonly cDNA is created with labeled primers for the RNA sequence of interest to act as the probe in the northern blot. The probes need to be labeled either with radioactive isotopes (e.g., ³²P) or with chemiluminescence in which alkaline phosphatase or horseradish peroxidase break down chemiluminescent substrates producing a detectable emission of light. The chemiluminescent labeling can occur in a couple of ways, the probe attached to the enzyme, or the probe labeled with a ligand (e.g. biotin) for which the antibody (e.g. avidin or streptavidin) is attached to the enzyme. X-ray film can detect both the radioactive and chemiluminescent signals.

Additional and alternative methods suitable for use in the method include Southern blotting and microarray hybridization, to name but two.

The assay is performed on a sample isolated from a subject. The sample can be any suitable source of relevant biological material such as cells, tissue, nucleic acid, protein, or any combination thereof. In one embodiment a sample is obtained as or from a bone marrow biopsy or bone marrow aspirate. In one embodiment a sample is obtained as or from a blood sample.

The method is useful to identify subjects at increased risk of mortality from B-ALL. As used herein, a subject “at increased risk of mortality from B-ALL” is a subject that has B-ALL and that has, to a statistically significant extent, a greater-than-average risk of mortality from the B-ALL. In one embodiment, a subject at increased risk of mortality from B-ALL is a subject that has B-ALL and that has at least a 10 percent greater-than-average risk of mortality from the B-ALL. In various embodiments, a subject at increased risk of mortality from B-ALL is a subject that has B-ALL and that has at least a 20 percent, at least a 30 percent, at least a 40 percent, or at least a 50 percent greater-than-average risk of mortality from the B-ALL.

In one embodiment, a subject at increased risk of mortality from B-ALL is a subject that has B-ALL and that has, to a statistically significant extent, a greater-than-average risk of relapse of the B-ALL. In one embodiment, a subject at increased risk of mortality from B-ALL is a subject that has B-ALL and that has at least a 10 percent greater-than-average risk of relapse the B-ALL. In various embodiments, a subject at increased risk of mortality from B-ALL is a subject that has B-ALL and that has at least a 20 percent, at least a 30 percent, at least a 40 percent, or at least a 50 percent greater-than-average risk of relapse of the B-ALL.

In one embodiment, a subject at increased risk of mortality from B-ALL is a subject that has B-ALL and that has, to a statistically significant extent, a less-than-average probability of disease-free survival. In one embodiment, a subject at increased risk of mortality from B-ALL is a subject that has B-ALL and that has at least a 10 percent less-than-average probability of disease-free survival. In various embodiments, a subject at increased risk of mortality from B-ALL is a subject that has B-ALL and that has at least a 20 percent, at least a 30 percent, at least a 40 percent, or at least a 50 percent less-than-average probability of disease-free survival.

In one embodiment, the method further includes the step of recording the result of performing the assay. The recording can be accomplished by any suitable means for recording a result and involve any suitable medium for recording the result. In one embodiment the recording is writing or printing the result onto paper or other tangible copy medium.

In another embodiment, the recording is electronically recording the result in a computer-readable medium, for example, on a hard drive, a flash drive, a compact disc (CD), or the like. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by client/server devices. Communication media typically embodies computer readable instructions, data structures, program modules or other data in a modulated data signal such as a transport mechanism and includes any information delivery media. “Modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above are included within the scope of computer readable media.

In yet another embodiment, the recording is recording an image of the assay result, such as in a photograph, photocopy, X-ray autoradiograph, digitized image, false-color digitized image, or the like.

The method for identifying subjects at increased risk of mortality from B-ALL may be coupled to a method for treating subjects so identified. That is, based on a result that identifies a subject as a subject at increased risk of mortality from B-ALL, the subject may be treated accordingly. For example, a subject identified as being at increased risk of mortality from B-ALL according to the method of the invention may be treated according to a method of treatment of the invention. Alternatively or in addition, a subject identified as being at increased risk of mortality from B-ALL according to the method of the invention may be treated early or even initially with a more aggressive or advanced type of therapy for B-ALL, such as bone marrow transplant or allogeneic stem cell transplant.

The invention also contemplates methods for identifying a subject that is not at increased risk of mortality from precursor B-cell acute lymphoblastic leukemia (B-ALL). In one embodiment the method includes the steps of performing an assay on a sample isolated from a subject, wherein the assay detects presence of mutant human cytokine receptor-like factor 2 (CRLF2) characterized by an amino acid mutation F232C, and identifying the subject as not having increased risk of mortality from B-ALL when the performing the assay does not detect the presence of the mutant CRLF2 in the sample.

In one embodiment the method includes the step of determining the absence of mutant human CRLF2 characterized by an amino acid mutation F232C by performing an assay on a sample isolated from a subject, wherein the absence of the mutant human CRLF2 in the sample indicates the subject is not at increased risk of mortality from B-ALL.

In one embodiment the method includes the step of detecting the absence of mutant human CRLF2 characterized by an amino acid mutation F232C in an assay performed on a sample isolated from a subject, wherein the absence of the mutant human CRLF2 in the sample indicates the subject is not at increased risk of mortality from B-ALL.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The therapeutics of the invention can be administered by any conventional route, including injection or by gradual infusion over time. The administration may, for example, be oral, intravenous, intratumoral, intraperitoneal, intramuscular, intracavity, subcutaneous, or transdermal.

The compositions of the invention are administered in effective amounts. An “effective amount” is that amount of an active agent (e.g., antisense, RNAi, or antibody that binds a mutant CRLF2 polypeptide) in a composition that alone, or together with further doses, produces the desired response, e.g. reduces expression or activity of the mutant CRLF2. In the case of treating B-ALL, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of a therapeutic agent (e.g., antisense, RNAi, or antibody that binds a mutant CRLF2 polypeptide) for producing the desired response in a unit of weight or volume suitable for administration to a patient. The response can, for example, be measured by determining the tumor burden following administration of the composition, such as regression of a tumor or decrease of disease symptoms. Other assays will be known to one of ordinary skill in the art and can be employed for measuring the level of the response.

The doses of compositions administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, compounds of the invention for use in the treatment of B-ALL are formulated and administered in doses between 1 ng and 1000 mg, and preferably between 10 ng and 1000 μg, according to any standard procedure in the art. Where nucleic acids are employed, doses of between 1 ng and 0.1 mg generally will be formulated and administered according to standard procedures. Other protocols for the administration of antibody-based compositions will be known to one of ordinary skill in the art, in which the dose amount, schedule of injections, sites of injections, mode of administration (e.g., intravenous) and the like vary from the foregoing. Administration of compositions to mammals other than humans, e.g. for testing purposes or veterinary therapeutic purposes, is carried out under substantially the same conditions as described above.

Where mutant CRLF2 polypeptides or immunogenic fragments thereof are used for vaccination, modes of administration that effectively deliver the polypeptide and adjuvant, such that an immune response to the polypeptide is increased, can be used. For administration of a polypeptide in adjuvant, methods include intradermal, intramuscular, subcutaneous, and intravenous administration. The invention is not limited by the particular modes of administration disclosed herein. Standard references in the art (e.g., Remington's Pharmaceutical Sciences, 18th edition, 1990) provide modes of administration and formulations for delivery of immunogens with adjuvant or in a non-adjuvant carrier.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, and lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases, and the like.

The pharmaceutical agents of the invention may be administered alone, in combination with each other, and/or in combination with other anti-cancer drug therapies and/or treatments. These therapies and/or treatments may include, but are not limited to: surgical intervention, chemotherapy, radiotherapy, and adjuvant systemic therapies.

The invention also provides a pharmaceutical kit comprising one or more containers comprising one or more of the pharmaceutical compounds or agents of the invention. Additional materials may be included in any or all kits of the invention, and such materials may include, but are not limited to buffers, water, enzymes, tubes, control molecules, etc. The kit may also include instructions for the use of the one or more pharmaceutical compounds or agents of the invention for the treatment of B-ALL.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### CRLF2 is a Gain-of-Function Oncoprotein in Poor Prognosis B-All

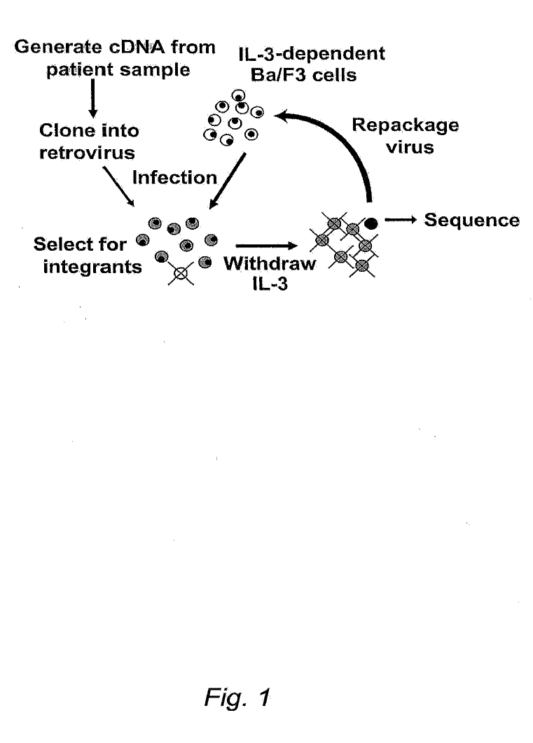

CRLF2 was identified in a functional screen for leukemia-derived cDNA that activate tyrosine kinase signaling (FIG. 1). In this screen, the murine IL-3 dependent cell line BaF3 were infected with retroviral cDNA libraries constructed from bone marrow aspirates involved with more than 80% tumor. Clones that survive IL3 withdrawal invariably harbor tumor-derived cDNAs that obviate the requirement for IL3. After infection with a cDNA library constructed from a B-ALL specimen with 46, XY karyotype, IL3-independent clones were identified that contained a mutated, full length cDNA transcript of CRLF2.

A combination of quantitative (q)RT-PCR, immunohistochemistry (IHC) and gene expression profiling (GEP) were used to assay CRLF2 expression in adult B-ALL samples from Dana-Farber Cancer Institute (DFCI; n=97) and Gruppo Malattie Ematologiche dell'Adulto (GIMEMA; n=157) cohorts. Cases with CRLF2 overexpression were clearly distinct, and correlated well between assays. Overall, CRLF2 was overexpressed in 15 (12.5%) of 120 adult B-ALL that lacked characteristic gene rearrangements, compared to 0 of 134 (0.0%) with these rearrangements (p<10⁻⁴). CRLF2 overexpression was not present in 69 cases of T-cell ALL assayed by gene expression profiling (p=0.001 compared to 15 of 120).

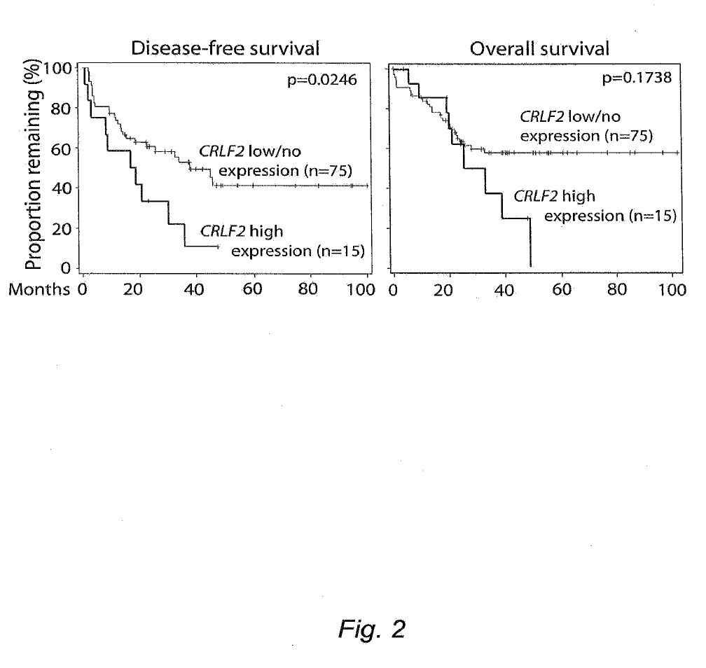

Ninety adult patients with B-ALL that lack characteristic rearrangements who had available demographic and outcome information, pooled from the DFCI (n=20) and GIMEMA (n=70) cohorts, were analyzed (Table 1). CRLF2 overexpressing and non-overexpressing cohorts had similar median age, sex distribution and white blood cell counts at presentation. However, disease-free survival (FIG. 2) was significantly shorter among patients with CRLF2 overexpression (median, 17.8 months versus 37.8 months; p<0.03). There was also a trend for shorter overall survival among these patients (median, 25.5 months versus not reached; p=0.17). Thus, CRLF2 overexpression is a marker for poor outcome among patients with B-ALL that lack characteristic rearrangements.

TABLE 1
CRLF2 Overexpression

| | Yes (n = 15), range | No (n = 75), range | p value |
|---|---|---|---|
| Median age (years) | 32.5, 19.3-67.1 | 34.3, 15.5-64.8 | 0.92 |
| WBC (×10³/μL) | 26.9, 1.9-422.0 | 20.2, 1.4-357.0 | 0.42 |
| Male:Female | 11:4 | 41:34 | 0.25 |
| Median overall | 25.5 months | Not reached | |
| survival | | | |
| Median disease-free | 17.8 months | 37.8 months | |
| survival | | | |

### Example 2

### CRLF2 Overexpression in Pediatric B-ALL

To determine the frequency of CRLF2 overexpression in pediatric B-ALL, the Gene Expression Omnibus (GEO; http://www.ncbi.nlm.nih.gov/geo/) database for B-ALL samples assayed on the Affymetrix U133 platform were reviewed. Affymetrix HG-U133A and HG-U133Aplus2 arrays contain a single probe set (208303_s_at) that targets the CRLF2 transcript (both complete and partial coding sequence, as well as expression sequence tags).

Nine databases (Table 2) with a total of 1,253 pediatric ALL cases were identified. Among the 3 databases that included only high-risk patients, 52 (14.8%) of 351 B-ALL that lacked characteristic rearrangements had CRLF2 overexpression compared with 0 (0.0%) of the remaining 130 B-ALLs (p<10⁻⁴). This may underestimate the frequency of CRLF2 overexpression in the cohort without characteristic rearrangements, as some databases did not distinguish patients based on karyotype, so patients with characteristic rearrangements were presumably included in the cohort of 351 B-ALLs.

In the six datasets that included both standard-risk and high-risk patients, CRLF2 overexpression was present in only 23 (4.1%) of 559 cases that lacked characteristic rearrangements (p<10⁻⁴ compared to 52 of 351 from the high-risk-only datasets). In addition, 0 (0.0%) of 51 cases of T-ALL had CRLF2 overexpression (p=0.002 compared to 52 of 351 from the high-risk-only datasets).

TABLE 2
Nine datasets of pediatric B-ALL specimens from the Gene
Expression Omnibus (http://www.ncbi.nlm.nih.gov/geo/).
A

| | High-risk | | | | | | CRLF2 overexpressing cases | | | |
|---|---|---|---|---|---|---|---|---|---|---|
| GEO ID | only | N | Focus | Median | SD | Cutoff | Focus | | Non-focus | |
| GSE10792 | No | 81 | 29 | 4.7696 | 0.31029 | 6.3211 | 1/29 | (3.45%) | 2/52 | (3.85%) |
| GSE13351 | No | 107 | 61 | 5.1766 | 0.21865 | 6.2698 | 4/61 | (6.56%) | 0/46 | (0.00%) |
| GSE13425 | No | 190 | 88 | 2.8183 | 0.26361 | 4.1363 | 6/88 | (6.82%) | 1/102 | (2.94%) |
| GSE635 | No | 173 | 173 | 2.7194 | 0.21644 | 3.8016 | 6/173 | (3.47%) | NA | |
| GSE10255 | No | 161 | 161 | 4.8546 | 0.22949 | 6.0021 | 4/161 | (2.48%) | NA | |
| GSE4698 | No | 60 | 47 | 6.6093 | 0.21097 | 7.6642 | 2/47 | (4.26%) | 0/13 | (0.00%) |
| | Total | 772 | 559 | | | | 23/559 | (4.11%) | 5/213 | (2.34%) |
| GSE12995 | Yes | 175 | 92 | 4.5694 | 0.25311 | 5.835 | 8/92 | (8.70%) | 0/83 | (0.00%) |
| GSE7440 | Yes | 99 | 99 | 4.5988 | 0.39049 | 6.5512 | 15/99 | (15.15%) | NA | |
| GSE11877 | Yes | 207 | 160 | 3.889 | 0.22982 | 5.0381 | 29/160 | (18.13%) | 0/47 | (0.00%) |
| | Total | 481 | 351 | | | | 52/351 | (14.81%) | 0/130 | (0.00%) |

B

| GEO ID | Category | Class Name | Focus group | Samples |
|---|---|---|---|---|
| GSE10792 | TEL-AML1 | CALL with t(12; 21) | No | 20 |
| GSE10792 | BCR-ABL | CALL-preB with t(9; 22) | No | 6 |
| GSE10792 | B-ALL | CALL-preB without t(9; 22) | Yes | 26 |
| GSE10792 | B-ALL | CALL-preB without t(9; 22) and TEL deleted | Yes | 3 |
| GSE10792 | MLL | prepreB-CALL with MLL | No | 26 |
| GSE13351 | T-ALL | T-ALL | No | 15 |
| GSE13351 | Hyperdiploidy | precursor-B ALL, subtype: Hyperdiploid | Yes | 28 |
| GSE13351 | BCR-ABL | precursor-B ALL, subtype: BCR-ABL | No | 1 |
| GSE13351 | E2A | precursor-B ALL, subtype: E2A-rearranged | No | 2 |
| GSE13351 | MLL | precursor-B ALL, subtype: MLL | No | 4 |
| GSE13351 | TEL-AML1 | precursor-B ALL, subtype: TEL-AML1 | No | 24 |
| GSE13351 | B-ALL | precursor-B ALL, subtype: other | Yes | 33 |
| GSE13425 | Hyperdiploidy | Precursor-B ALL, subtype: Hyperdiploid | Yes | 44 |
| GSE13425 | BCR-ABL | Precursor-B ALL, subtype: BCR-ABL | No | 4 |
| GSE13425 | BCR-ABL | Precursor-B ALL, subtype: BCR-ABL | No | 1 |
| | | (+hyperdiploidy) | | |
| GSE13425 | E2A | Precursor-B ALL, subtype: E2A-rearranged (E) | No | 1 |
| GSE13425 | E2A | Precursor-B ALL, subtype: E2A-rearranged (E- | No | 4 |
| | | sub) | | |
| GSE13425 | E2A | Precursor-B ALL, subtype: E2A-rearranged | No | 8 |
| | | (EP) | | |
| GSE13425 | MLL | Precursor-B ALL, subtype: MLL | No | 4 |
| GSE13425 | TEL-AML1 | Precursor-B ALL, subtype: TEL-AML1 | No | 43 |
| GSE13425 | TEL-AML1 | Precursor-B ALL, subtype: TEL-AML1 | No | 1 |
| | | (+hyperdiploidy) | | |
| GSE13425 | B-ALL | Precursor-B ALL, subtype: other | Yes | 44 |
| GSE13425 | T-ALL | T-ALL | No | 36 |
| GSE635 | B-ALL | primary acute lymphoblastic leukemia cells | Yes | 173 |
| GSE10255 | B-ALL | ALL cells from pediatric diagnostic bone | Yes | 161 |
| | | marrow, methotrexate treated | | |
| GSE4698 | BCR-ABL | Genetic features: BCR-ABL positive | No | 2 |
| GSE4698 | E2A | Genetic features: E2A-PBX1 positive | No | 1 |
| GSE4698 | MLL | Genetic features: MLL-AF4 positive | No | 3 |
| GSE4698 | TEL-AML1 | Genetic features: TEL-AML1 positive | No | 7 |
| GSE4698 | Hyperdiploidy | Genetic features: hyperdiploid | Yes | 7 |
| GSE4698 | B-ALL | Genetic features: not found | Yes | 40 |
| GSE12995 | BCR-ABL | B-progenitor ALL with BCR-ABL1 fusion | No | 20 |
| GSE12995 | TEL-AML1 | B-progenitor ALL with ETV6-RUNX1 fusion | No | 34 |
| GSE12995 | MLL | B-progenitor ALL with MLL rearrangement | No | 13 |
| GSE12995 | E2A | B-progenitor ALL with TCF3-PBX1 fusion | No | 16 |
| GSE12995 | Hyperdiploidy | B-progenitor ALL with high hyperdiploidy (47- | Yes | 16 |
| | | 50 chromosomes) | | |
| GSE12995 | Hyperdiploidy | B-progenitor ALL with high hyperdiploidy (>50 | Yes | 28 |
| | | chromosomes) | | |
| GSE12995 | Hypodiploidy | B-progenitor ALL with hypodiploidy | Yes | 7 |
| GSE12995 | B-ALL | B-progenitor ALL with normal chromosome | Yes | 16 |
| | | number but structural rearrangements | | |
| GSE12995 | B-ALL | Miscellaneous B-progenitor ALL | Yes | 25 |
| GSE7440 | B-ALL | CCR: continuous complete remission for >4 | Yes | 8 |
| | | years | | |
| GSE7440 | B-ALL | RER: rapid early responder = patient with M1 | Yes | 23 |
| | | marrow at Day 7 | | |
| GSE7440 | B-ALL | RER: rapid early responder = patient with M1 | Yes | 11 |
| | | marrow at Day 7; CCR: continuous complete | | |
| | | remission for >4 years | | |
| GSE7440 | B-ALL | RER: rapid early responder = patient with M1 | Yes | 8 |
| | | marrow at Day 7; Relapse: patients that | | |
| | | relapsed within 3 years of initial diagnosis | | |
| GSE7440 | B-ALL | Relapse: patients that relapsed within 3 years | Yes | 9 |
| | | of initial diagnosis | | |
| GSE7440 | B-ALL | SER: slow early responder = patient with M3 | Yes | 17 |
| | | marrow at Day 7 | | |
| GSE7440 | B-ALL | SER: slow early responder = patient with M3 | Yes | 9 |
| | | marrow at Day 7, CCR: continuous complete | | |
| | | remission for >4 years | | |
| GSE7440 | B-ALL | SER: slow early responder = patient with M3 | Yes | 14 |
| | | marrow at Day 7; Relapse: patients that | | |
| | | relapsed within 3 years of initial diagnosis | | |
| GSE11877 | MLL | MLL: Positive | | 21 |
| GSE11877 | TEL-AML1 | TEL-AML, t(12; 21): Positive | | 3 |
| GSE11877 | Hyperdiploidy | trisomy 4 and 10: Positive | | 5 |
| GSE11877 | E2A | E2A-PBX, t(1; 19): Positive | | 23 |
| GSE11877 | BCR-ABL | BCR-ABL, t(9; 22): Positive | | 0 |
| GSE11877 | B-ALL | All Negative | | 155 |

All specimens were analyzed with Affymetrix U133A or U133A Plus 2.0 arrays and CRLF2 expression was quantified based on signal from the 208303_s_at probe. A. The Focus set includes only cases of B-ALL that lack TEL/AML, MLL, E2A, or BCR/ABL rearrangements. Cases reported to be T-ALL (n = 51) were also excluded from the Focus set. The cutoff for CRLF2 overexpression was defined individually for each data set as 5 standard deviations above the median of the background distribution. The definition of high-risk for the three sets that only included high-risk cases varied between datasets. B. Cohorts based on lineage and gene rearrangements.

### Example 3

### CRLF2 in Other Common Lymphoid Malignancies

In order to determine whether CRLF2 is overexpressed in other lymphoid malignancies, a cross-section of chronic lymphocytic leukemia (CLL) specimens, based on karyotype, IgV_{H} somatic hypermutation, ZAP-70 and CD38 expression, CLL family history, and clinical features were selected. All thirty specimens had no detectable CRLF2 mRNA. A review of gene expression profile data from GEO dataset GSE6477 (n=162) failed to identify significant CRLF2 expression in normal plasma cells, monoclonal gammopathy of undetermined significance, smoldering multiple myeloma (MM), newly diagnosed MM or relapsed MM. Low or undetectable CRLF2 expression was also confirmed in a panel of T-cell ALL (n=22) and other (n=14) cell lines.

### Example 4

### CRLF2 Locus Rearrangements

Russell et al. (2009) Blood 114:2688-98 recently demonstrated that the CRLF2 locus, which is located in the pseudoautosomal regions of chromosomes X and Y, can undergo two types of rearrangement, intrachromosomal deletion or translocation with IGH. In both cases, the chr.X/Y breakpoints are upstream of CRLF2 and place the full CRLF2 coding sequence under the control of alternate transcriptional control elements.

CRLF2 and IGH are close to the telomeres of chr.X/Y and 14, respectively. Thus, a FISH strategy using probes against regions flanking CRLF2 and IGH was designed. While the CRLF2 and IGH loci are clearly separate in cells that lack CRLF2 expression, FISH in 3 of 6 B-ALL specimens with high CRLF2 expression demonstrated joining of CRLF2 and IGH probes, consistent with a reciprocal chromosomal translocation. Two of the three remaining specimens had loss of a centromeric chr. X/Y probe, consistent with an intrachromosomal deletion. The final specimen had neither a deletion nor a translocation, suggesting an additional mechanism for CRLF2 overexpression.

CRLF2/IGH translocations are akin to IGH rearrangements with MYC, BCL2, and BCL1 that result from aberrant V(D)J recombination. In 6 specimens, der(14) translocation junctions between IgHJ segments on chr.14 and the region centromeric of CRLF2 on chr.X/Y were PCR amplified. Junctions involved sequence approximately 8-16 kb upstream of the CRLF2 translation start site, with multiple cases clustering near putative V(D)J recombinase recognition signal sequences. Thus, CRLF2/IGH translocations appear to result from aberrant V(D)J recombination that can involve cryptic recognition signal sequences in the pseudoautosomal regions.

### Example 5

### CRLF2 Phe232Cys is a Gain-of-Function Mutation

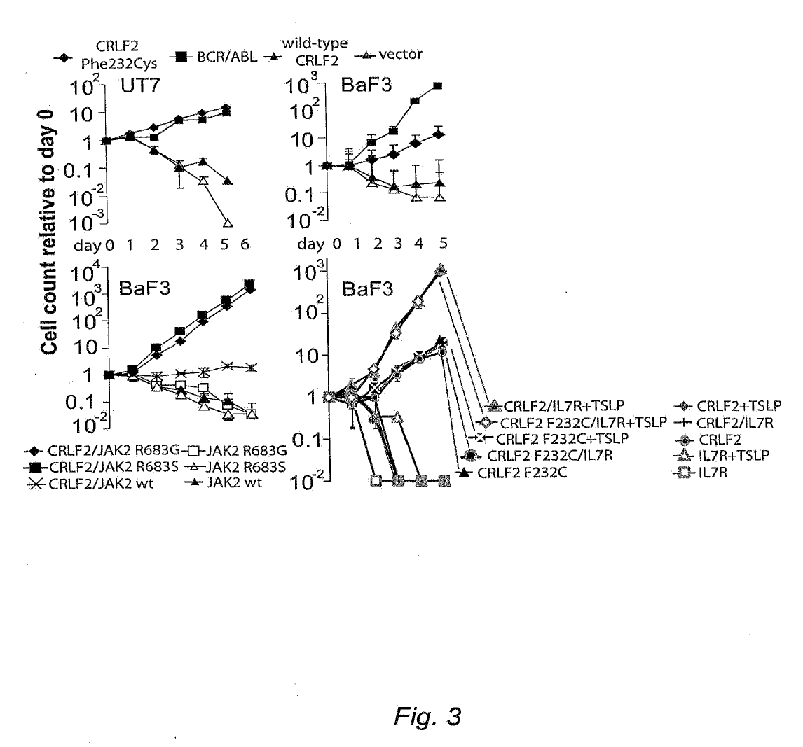

Sequencing of CRLF2 in 35 B-ALL specimens, including 14 overexpressing cases, identified multiple single nucleotide variants. The function of the 4 nonsynonymous variants (711T>G, 746G>A, 789A>G, 984C>T) were assayed by retroviral expression in BaF3 cells. Of these, only CRLF2 711T>G (Phe232Cys) conferred cytokine independence in murine BaF3 and human UT7 megakaryoblastic leukemia cells (FIG. 3). The 711T>G mutation was present in 3 (21.4%) of 14 overexpressing cases and was a somatic mutation. Sequencing of cDNA from all three cases demonstrated expression of the mutant allele. The other three nonsynonymous variants were polymorphisms, as they were present in germline specimens and were recovered from healthy donor lymphocytes.

The CRLF2 Phe232 residue is near the junction of the extracellular and transmembrane domains. Mutations that introduce cysteine residues in this region of other receptor tyrosine kinases, such as RET, can activate signal transduction through intermolecular disulfide-bonded dimers. To confirm that CRLF2 Phe232Cys promotes constitutive dimerization, immunoblots in BaF3 cells expressing wild-type CRLF2 or CRLF2 Phe232Cys were performed under both reducing and non-reducing conditions. Under non-reducing conditions, the molecular weight of the CRLF2 Phe232Cys band, but not the wild-type band, was doubled, consistent with constitutive dimerization through the cysteine residues.

### Example 6

### JAK2 Mutations are Highly Associated with CRLF2 Overexpression

The absence of gain-of-function CRLF2 mutations in most cases with CRLF2 overexpression raised the possibility that other factors within the same signaling cascade may harbor mutations. JAK mutations were recently reported in children with B-ALL. Sequencing for previously identified mutations in JAK1 and JAK2, JAK2 Arg683Gly (n=4), Arg683Ser (n=1), and Arg683Thr (n=1) substitutions were identified in 6 of 14 adult B-ALL cases that overexpress wild-type CRLF2. Of note, expression of CRLF2 Phe232Cys and JAK2 mutant alleles was mutually exclusive, suggesting that they function within the same pathway.

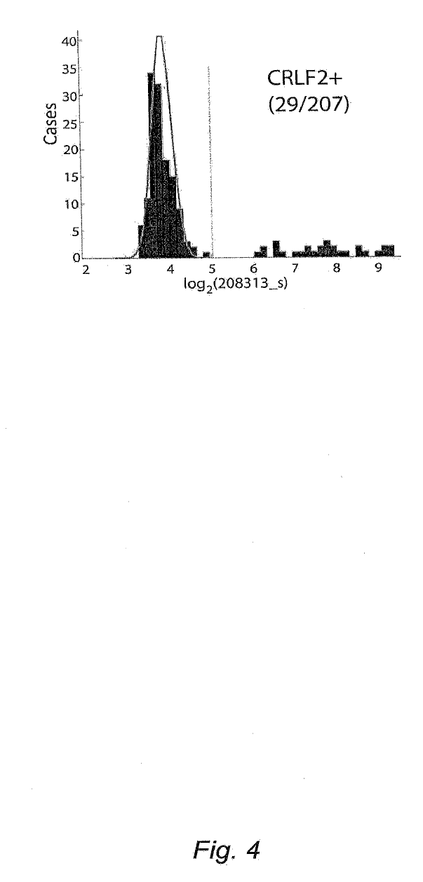

Mutant JAK proteins can only transform growth factor-dependent cells when expressed in combination with a type I cytokine receptor. Bercovich et al. (2008) Lancet 372:1484-92. The specific receptor that the JAK associates with, along with the particular JAK mutation, can affect the transformed phenotype. Mullighan (2008) Lancet 372:1448-50. Thus, analysis was performed to determine whether overexpression of CRLF2 is essential for B-ALL associated with mutant JAK2. Gene expression (GEO #GSE11877) and JAK mutation status from a cohort of 207 patients with high-risk pediatric B-ALL were linked CRLF2 expression among the 207 patients was clearly bimodal, with overexpression in 29 (14.0%) cases (FIG. 4). Twenty of the 207 patients also had mutations in JAK1 (n=3), JAK2 (n=16) or JAK3 (n=1). Mullighan et al. (2009) Proc Natl Acad Sci USA 106:9414-8. Strikingly, all 16 (100%) patients with JAK2 exon 16 mutations overexpressed CRLF2, as did two of the three patients with JAK1 mutations.

### Example 7

### CRLF2 and JAK2 Contribute to Cytokine-Independent Growth

The finding that all cases of B-ALL with JAK2 mutations overexpressed CRLF2 raised the possibility that JAK2 associates directly with CRLF2, either in the presence or the absence of IL-7R. The stoichiometry of a wild-type CRLF2/IL-7R complex is believed to be 1:1. Pandey et al. (2000) Nat Immunol 1:59-64. RT-PCR and gene expression profiling demonstrated that IL-7R expression did not differ between the CRLF2 overexpressing and non-overexpressing cases. This suggests that mutated JAK2 signals with CRLF2 in the absence of IL-7R. To test this possibility, wild-type and mutant versions of CRLF2 and JAK2 were co-expressed in the presence or absence of IL-7R in BaF3 cells (FIG. 3). While wild-type CRLF2 or mutant JAK2 alone were insufficient to confer IL3 independence in BaF3 cells, the combination readily transformed the cells to IL3-independent growth in the absence of IL-7R. Of note, the combination of wild-type CRLF2 with wild-type JAK2 provided a growth advantage over JAK2 alone (FIG. 3).

Unlike cells that express CRLF2/IL-7R, the addition of TSLP had no effect on the growth of BaF3 cells expressing CRLF2 Phe232Cys (FIG. 3). However, TSLP promoted the growth of cells that express CRLF2 Phe232Cys/IL-7R (FIG. 3), suggesting that CRLF2 Phe232Cys retains the ability to signal within a heterodimer. In contrast, TSLP did not promote the growth of cells that express CRLF2/mutant JAK2.

In BaF3 cells, both CRLF2 Phe232Cys and CRLF2/mutant JAK2 promoted the phosphorylation of the JAK targets STAT5 and ERK. Phosphorylation of STAT5 bp CRLF2 Phe232Cys was less robust than by CRLF2/mutant JAK2 but comparable to CRLF2/IL-7R/TSLP. CRLF2 Phe232Cys also promoted the upregulation of transcriptional targets downstream of JAKs (BCL-xL and PIM1) to a comparable or greater extent than wild-type CRLF2/mutant JAK2.

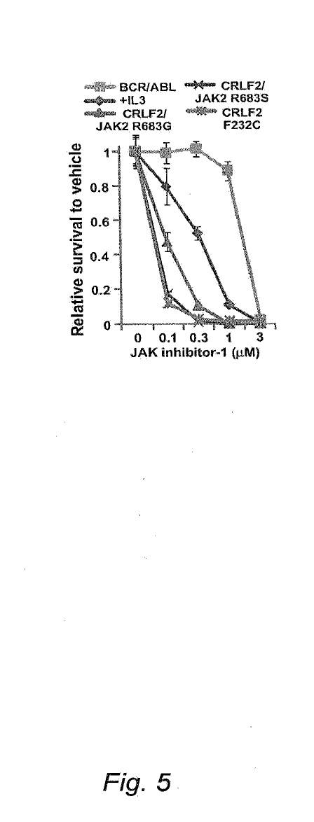

While cells expressing wild-type CRLF2/mutant JAK2 had constitutively phosphorylated JAK2, cells expressing CRLF2 Phe232Cys and CRLF2/IL-7R cells treated with TSLP had no detectable phospho-JAK2. Yet, cells expressing either the CRLF2 Phe232Cys or CRLF2/mutant JAK2 were highly sensitive to a small molecule JAK inhibitor (FIG. 5), suggesting that CRLF2 signaling involves a JAK other than JAK2. As expected, TSLP had no effect on STAT5, ERK or JAK2 phosphorylation in cells with mutant CRLF2 or JAK2 proteins.

### Example 8

### CRLF2 Transcriptional Signature in Adult and Pediatric B-All

To characterize the dysregulated genes associated with CRLF2 overexpression, a supervised analysis of gene expression profiles from the 22 B-ALL (8 CRLF2 overexpressing, 14 non-overexpressing) that were validated by qRT-PCR was performed. CRLF2 overexpression defined a 130 probe set (105 gene) “CRLF2 adult signature” (Table 3). Upregulated genes in the CRLF2 adult signature included CD10, protein kinase C (PKC) iota, and the STAT5-induced negative regulator SOCS2, while genes showing decreased expression included the class I and class II human leukocyte antigens. Higher SOCS2 expression among CRLF2 overexpressing cases was confirmed by qRT-PCR (123.1-fold higher than donor peripheral blood leukocytes (PBLs) vs. 53.5-fold for CRLF2 non-overexpressing cases; p<0.05). Interestingly, four agents (Go6976, UCN-01, PKC412 and k252a) with activity against PKC family kinases had selective toxicity in BaF3-CRLF2 Phe232Cys cells, as compared to BaF3 cells expressing wild-type CRLF2.

TABLE 3
CRLF2 adult gene expression signature.

| | | | | Lower Bound | Upper Bound | Difference | | |
|---|---|---|---|---|---|---|---|---|
| Gene | Probe Set | Accession | FC | of FC | of FC | of Means | t stat | P value |
| cytokine receptor-like | 208303_s_at | NM_022148 | 17.63 | 11.84 | 24.25 | −986.26 | −4.972 | 0.001608 |
| factor 2 | | | | | | | | |
| CD10/CALLA | 203434_s_at | AI433463 | 3.77 | 2.16 | 9.79 | −2459.66 | −4.079 | 0.001318 |
| lymphoid enhancer- | 210948_s_at | AF294627 | 3.53 | 2.19 | 6.16 | −1008.98 | −3.777 | 0.004282 |
| binding factor 1 | | | | | | | | |
| CD10/CALLA | 203435_s_at | NM_007287 | 3.04 | 1.77 | 7.08 | −3466.7 | −3.441 | 0.004072 |
| CDNA FLJ37485 | 1558517_s_at | CA773938 | 2.84 | 2.04 | 4.07 | −206.66 | −4.766 | 0.000735 |
| SERPINB9 | 1563357_at | AL049245 | 2.82 | 2.03 | 4.12 | −129.86 | −4.859 | 0.000518 |
| ALDH5A1 | 203608_at | AL031230 | 2.68 | 1.77 | 4.25 | −488.52 | −3.588 | 0.004703 |
| chr 18 open reading | 207996_s_at | NM_004338 | 2.65 | 1.74 | 4.35 | −647.19 | −3.58 | 0.004212 |
| frame 1 | | | | | | | | |
| hypothetical protein | 218532_s_at | NM_019000 | 2.6 | 1.91 | 3.94 | −333.67 | −6.062 | 0.000007 |
| FLJ20152 | | | | | | | | |
| FLJ37858 protein | 227354_at | BF589359 | 2.56 | 1.8 | 3.88 | −867.87 | −4.211 | 0.001218 |
| HIVEP2 | 212642_s_at | AL023584 | 2.43 | 1.73 | 3.6 | −362.88 | −4.289 | 0.000886 |
| adducin 3 (gamma) | 201034_at | BE545756 | 2.42 | 1.62 | 3.97 | −1472.56 | −3.556 | 0.00346 |
| GALNT7 | 218313_s_at | NM_017423 | 2.42 | 1.76 | 3.52 | −1004.39 | −4.583 | 0.000453 |
| lymphoid enhancer- | 221558_s_at | AF288571 | 2.4 | 1.67 | 3.81 | −2793.08 | −4.102 | 0.000888 |
| binding factor 1 | | | | | | | | |
| LRIG1 | 211596_s_at | AB050468 | 2.36 | 1.62 | 3.6 | −898.53 | −3.595 | 0.003874 |
| CDNA FLJ37485 | 228314_at | BE877357 | 2.25 | 1.63 | 3.14 | −466.81 | −3.805 | 0.003256 |
| KIAA0582 | 212675_s_at | AB011154 | 2.18 | 1.6 | 2.98 | −917.99 | −3.822 | 0.00321 |
| PAG1 | 225626_at | AK000680 | 2.14 | 1.71 | 2.74 | −2701.52 | −5.614 | 0.000057 |
| Solute carrier family 38, | 224579_at | BF247552 | 2.13 | 1.71 | 2.73 | −2188.38 | −5.784 | 0.000034 |
| member 1 | | | | | | | | |
| chr 10 open reading | 225785_at | BG112359 | 2.12 | 1.53 | 3.1 | −276.59 | −3.814 | 0.001707 |
| frame 74 | | | | | | | | |
| adducin 3 (gamma) | 201752_s_at | AI763123 | 2.1 | 1.52 | 3.1 | −620.38 | −3.857 | 0.001401 |
| adducin 3 (gamma) | 205882_x_at | AI818488 | 2.09 | 1.52 | 3.05 | −597.18 | −3.963 | 0.001051 |
| hypothetical protein | 218510_x_at | AI816291 | 2.09 | 1.56 | 3.04 | −306.15 | −4.652 | 0.000155 |
| FLJ20152 | | | | | | | | |
| zinc and ring finger 2 | 226261_at | AI831561 | 2.05 | 1.51 | 2.84 | −102.19 | −3.696 | 0.002994 |
| chr2 synaptotagmin | 224698_at | AB033054 | 2.04 | 1.6 | 2.62 | −1056.52 | −4.666 | 0.000529 |
| EGF-like-domain, multiple | 212830_at | W68084 | 2 | 1.46 | 2.78 | −215.24 | −3.473 | 0.004595 |
| 5 | | | | | | | | |
| Full length insert cDNA | 228478_at | AA889954 | 1.99 | 1.57 | 2.57 | −103.95 | −4.717 | 0.000341 |
| YH99G08 | | | | | | | | |
| Solute carrier family 38, | 224580_at | BF515894 | 1.98 | 1.47 | 2.73 | −146.14 | −3.608 | 0.003377 |
| member 1 | | | | | | | | |
| chr 16 open reading | 1559584_a_at | BC025741 | 1.95 | 1.47 | 2.74 | −398.17 | −3.964 | 0.001004 |
| frame 54 | | | | | | | | |
| chr 10 open reading | 227701_at | AK024739 | 1.94 | 1.5 | 2.59 | −204.67 | −4.257 | 0.000664 |
| frame 118 | | | | | | | | |
| Solute carrier family 38, | 218237_s_at | NM_030674 | 1.9 | 1.41 | 2.7 | −1731.18 | −3.712 | 0.001621 |
| member 1 | | | | | | | | |
| zinc finger protein 85 | 206572_x_at | NM_003429 | 1.86 | 1.45 | 2.36 | −490.45 | −3.82 | 0.003157 |
| (HPF4, HTF1) | | | | | | | | |
| speckle-type POZ protein | 208927_at | BF673888 | 1.86 | 1.43 | 2.56 | −417.41 | −4.07 | 0.000652 |
| protein kinase C, iota | 209678_s_at | L18964 | 1.86 | 1.4 | 2.56 | −187.6 | −3.594 | 0.002551 |
| hypothetical protein | 238510_at | AA744964 | 1.85 | 1.38 | 2.61 | −133.75 | −3.5 | 0.002788 |
| LOC124411 | | | | | | | | |
| MYB | 204798_at | NM_005375 | 1.82 | 1.39 | 2.47 | −2090 | −3.629 | 0.002358 |
| TLE4 | 204872_at | NM_007005 | 1.81 | 1.39 | 2.41 | −708.03 | −3.655 | 0.002542 |
| Cbl-interacting protein | 238587_at | AI927919 | 1.81 | 1.32 | 2.61 | −715.6 | −3.222 | 0.004723 |
| Sts-1 | | | | | | | | |
| CD99 antigen | 201029_s_at | NM_002414 | 1.78 | 1.36 | 2.52 | −3827.11 | −4.09 | 0.000796 |
| translin-associated factor | 203983_at | NM_005999 | 1.78 | 1.39 | 2.34 | −338.73 | −3.856 | 0.00142 |
| X | | | | | | | | |
| HIBCH | 213374_x_at | AW000964 | 1.77 | 1.46 | 2.15 | −123.71 | −4.635 | 0.000546 |
| CREB1 | 204314_s_at | NM_004379 | 1.75 | 1.34 | 2.37 | −202.8 | −3.467 | 0.002995 |
| MTF2 | 203345_s_at | AI566096 | 1.7 | 1.33 | 2.17 | −515.51 | −3.45 | 0.004816 |
| tetraspanin 13 | 217979_at | NM_014399 | 1.69 | 1.33 | 2.2 | −1351.55 | −3.586 | 0.002415 |
| UPF3B | 218757_s_at | NM_023010 | 1.66 | 1.28 | 2.23 | −365.34 | −3.363 | 0.003217 |
| selenophosphate | 200961_at | NM_012248 | 1.63 | 1.32 | 2.02 | −365.34 | −3.698 | 0.00241 |
| synthetase 2 | | | | | | | | |
| PBXIP1 | 214176_s_at | AI348545 | 1.63 | 1.29 | 2.08 | −176.27 | −3.387 | 0.004102 |
| suppressor of cytokine | 203373_at | NM_003877 | 1.62 | 1.29 | 2.15 | −3260.3 | −3.753 | 0.001393 |
| signaling 2 | | | | | | | | |
| helicase with zinc finger | 203674_at | NM_014877 | 1.62 | 1.28 | 2.08 | −286.83 | −3.352 | 0.004089 |
| domain | | | | | | | | |
| KIAA0143 protein | 212149_at | AW470003 | 1.61 | 1.3 | 2.01 | −243.46 | −3.684 | 0.002192 |
| LRP5 | 209468_at | AB017498 | 1.6 | 1.35 | 1.88 | −105.77 | −4.088 | 0.002801 |
| PDHX | 203067_at | NM_003477 | 1.59 | 1.26 | 2.06 | −164 | −3.289 | 0.00419 |
| WAS protein family, | 224562_at | AK025566 | 1.57 | 1.28 | 1.96 | −1395.67 | −3.595 | 0.002332 |
| member 2 | | | | | | | | |
| fetal Alzheimer antigen | 232909_s_at | AU146870 | 1.55 | 1.24 | 2 | −210.17 | −3.258 | 0.004207 |
| MBD4 | 214047_s_at | AI913365 | 1.53 | 1.25 | 1.89 | −181.17 | −3.519 | 0.002781 |
| chr 6 open reading frame | 213322_at | AL031778 | 1.5 | 1.24 | 1.83 | −116.58 | −3.561 | 0.002472 |
| 130 | | | | | | | | |
| Ferritin, light polypeptide | 213187_x_at | BG538564 | −1.5 | −1.23 | −1.87 | 1897.49 | 3.374 | 0.003217 |
| SUMO3 | 200740_s_at | NM_006936 | −1.53 | −1.24 | −1.93 | 686.41 | 3.314 | 0.003687 |
| CDC37 | 209953_s_at | U63131 | −1.53 | −1.27 | −1.87 | 185.42 | 3.708 | 0.001416 |
| ubiquitin-conjugating | 201523_x_at | BE262760 | −1.54 | −1.24 | −1.95 | 200.39 | 3.276 | 0.004046 |
| enzyme E2N | | | | | | | | |
| NONO | 208698_s_at | L14599 | −1.54 | −1.24 | −1.93 | 352.52 | 3.369 | 0.00318 |
| PSMC3 | 201267_s_at | AL545523 | −1.56 | −1.26 | −1.95 | 153.22 | 3.412 | 0.002773 |
| fibrillarin | 211623_s_at | M30448 | −1.56 | −1.25 | −1.97 | 1144.42 | 3.231 | 0.004187 |
| DYNC1LI1 | 222479_s_at | AK001081 | −1.56 | −1.32 | −1.86 | 136.57 | 4.2 | 0.000441 |
| SKI interacting protein | 215424_s_at | AV689564 | −1.57 | −1.32 | −1.83 | 219.04 | 4.062 | 0.000794 |
| Myelin protein zero-like 1 | 210594_x_at | AF239756 | −1.58 | −1.32 | −1.88 | 100.76 | 3.936 | 0.000916 |
| BCL2-associated | 211475_s_at | AF116273 | −1.59 | −1.29 | −1.98 | 286.69 | 3.6 | 0.001788 |
| athanogene | | | | | | | | |
| HNRPDL | 1554678_s_at | AB066484 | −1.59 | −1.36 | −1.88 | 544.51 | 5.121 | 0.000094 |
| solute carrier family 25, | 221432_s_at | NM_031212 | −1.6 | −1.27 | −2.04 | 146.01 | 3.286 | 0.003698 |
| member 28 | | | | | | | | |
| GNB1 | 200744_s_at | AI741124 | −1.61 | −1.33 | −1.98 | 274.48 | 4.156 | 0.000565 |
| deoxyribonuclease I | 1558546_at | BM802340 | −1.61 | −1.29 | −1.97 | 102.19 | 3.311 | 0.004196 |
| CDNA FLJ38849 | 221877_at | BF508835 | −1.63 | −1.3 | −2.09 | 378.68 | 3.608 | 0.001868 |
| GOLGA7 | 1554167_a_at | BC012032 | −1.65 | −1.3 | −2.14 | 175.63 | 3.363 | 0.003096 |
| ILF2 | 200052_s_at | NM_004515 | −1.66 | −1.32 | −2.12 | 489 | 3.668 | 0.001548 |
| SLC25A6 | 212085_at | AA916851 | −1.66 | −1.3 | −2.23 | 2140.75 | 3.563 | 0.002677 |
| zinc finger protein 317 | 225296_at | AB046808 | −1.66 | −1.32 | −2.1 | 128.79 | 3.591 | 0.001829 |
| hypothetical protein | 218156_s_at | NM_018128 | −1.67 | −1.36 | −2.07 | 111.78 | 4.078 | 0.00059 |
| FLJ10534 | | | | | | | | |
| aldolase C, fructose- | 202022_at | NM_005165 | −1.68 | −1.37 | −2.1 | 116.33 | 4.189 | 0.000466 |
| bisphosphate | | | | | | | | |
| HLA-G, class I, G | 211528_x_at | M90685 | −1.68 | −1.31 | −2.17 | 2003.06 | 3.35 | 0.003225 |
| hypothetical protein | 221807_s_at | BG399562 | −1.7 | −1.39 | −2.1 | 123.56 | 4.117 | 0.000552 |
| PP2447 | | | | | | | | |
| SUMO3 | 200739_s_at | BG338532 | −1.72 | −1.39 | −2.14 | 159.79 | 3.984 | 0.000761 |
| phosphogluconate | 201118_at | NM_002631 | −1.72 | −1.35 | −2.24 | 182.33 | 3.716 | 0.001388 |
| dehydrogenase | | | | | | | | |
| DDX21 | 208152_s_at | NM_004728 | −1.72 | −1.37 | −2.19 | 948.15 | 3.762 | 0.001236 |
| CDNA FLJ38849 | 64488_at | AW003091 | −1.72 | −1.32 | −2.34 | 307.03 | 3.509 | 0.002606 |
| RAPH1 | 204346_s_at | NM_007182 | −1.75 | −1.35 | −2.31 | 232.8 | 3.416 | 0.002755 |
| EEF1A1 | 227708_at | AW469790 | −1.75 | −1.35 | −2.26 | 359.43 | 3.382 | 0.003191 |
| clone 38C16 on chr | 234954_at | AL035604 | −1.76 | −1.48 | −2.1 | 141.92 | 4.953 | 0.000093 |
| 6q22.33-24.1 | | | | | | | | |
| HLA-G, class I, G | 211529_x_at | M90684 | −1.77 | −1.34 | −2.36 | 1971.46 | 3.274 | 0.003828 |
| DDX21 | 224654_at | BG164358 | −1.78 | −1.4 | −2.29 | 1040.71 | 3.764 | 0.001258 |
| DDX24 | 200694_s_at | NM_020414 | −1.79 | −1.45 | −2.18 | 642.16 | 4.115 | 0.000721 |
| PTBP1 | 212016_s_at | AA679988 | −1.8 | −1.38 | −2.32 | 155.05 | 3.381 | 0.003367 |
| UQCRC2 | 200883_at | NM_003366 | −1.81 | −1.43 | −2.35 | 337.91 | 4.232 | 0.000428 |
| Coronin, actin binding | 222409_at | AL162070 | −1.83 | −1.41 | −2.38 | 288.87 | 3.623 | 0.001827 |
| protein, 1C | | | | | | | | |
| NR1H2 | 218215_s_at | NM_007121 | −1.86 | −1.37 | −2.64 | 166.69 | 3.371 | 0.003074 |
| QTRT1 | 221270_s_at | NM_031209 | −1.87 | −1.43 | −2.57 | 113.32 | 4.024 | 0.000791 |
| DNAJC4 | 206782_s_at | NM_005528 | −1.9 | −1.5 | −2.48 | 157.75 | 4.775 | 0.000148 |
| TIMM23 | 218119_at | NM_006327 | −1.9 | −1.52 | −2.41 | 111.2 | 4.737 | 0.000126 |
| ubiquitin-conjugating | 200669_s_at | NM_003340 | −1.91 | −1.44 | −2.64 | 1628.73 | 3.809 | 0.001132 |
| enzyme E2D 3 | | | | | | | | |
| BCL2-associated | 202387_at | NM_004323 | −1.91 | −1.46 | −2.48 | 376.99 | 3.654 | 0.001871 |
| athanogene | | | | | | | | |
| PRO0470 | 220856_x_at | NM_014128 | −1.94 | −1.44 | −2.72 | 709.14 | 3.657 | 0.001568 |
| Catalase | 211922_s_at | AY028632 | −1.95 | −1.46 | −2.55 | 320.94 | 3.432 | 0.003298 |
| MHC, class II, DR alpha | 208894_at | M60334 | −1.97 | −1.47 | −2.73 | 3854.81 | 3.749 | 0.001264 |
| ferritin, heavy | 200748_s_at | NM_002032 | −2.02 | −1.47 | −2.78 | 4685.94 | 3.441 | 0.002821 |
| polypeptide 1 | | | | | | | | |
| DYNLT1 | 201999_s_at | NM_006519 | −2.03 | −1.43 | −3 | 856.65 | 3.251 | 0.004038 |
| ADP-ribosylation factor 1 | 208750_s_at | AA580004 | −2.03 | −1.62 | −2.59 | 380.2 | 5.376 | 0.00003 |
| CDNA FLJ13267 | 212829_at | BE878277 | −2.07 | −1.57 | −2.73 | 270.88 | 3.922 | 0.001025 |
| G3BP2 | 206383_s_at | NM_012297 | −2.09 | −1.49 | −2.89 | 106.61 | 3.288 | 0.004293 |
| G3BP2 | 208840_s_at | AU149503 | −2.09 | −1.49 | −3.02 | 367.88 | 3.406 | 0.002909 |
| ras homolog gene family, | 1555814_a_at | AF498970 | −2.14 | −1.69 | −2.71 | 1591.53 | 4.882 | 0.000121 |
| member A | | | | | | | | |
| hypothetical protein | 224916_at | BG110811 | −2.19 | −1.47 | −3.59 | 160.42 | 3.238 | 0.004124 |
| LOC340061 | | | | | | | | |
| cartilage associated | 1554464_a_at | BC008745 | −2.19 | −1.58 | −2.99 | 150.78 | 3.509 | 0.002788 |
| protein | | | | | | | | |
| ribosomal protein L10 | 221989_at | AW057781 | −2.21 | −1.51 | −3.51 | 486.84 | 3.411 | 0.002774 |
| P4HB | 1564494_s_at | AK075503 | −2.22 | −1.52 | −3.48 | 162.7 | 3.425 | 0.002691 |
| HLA-G, class I, G | 210514_x_at | AF226990 | −2.3 | −1.69 | −3.16 | 1469.32 | 4.073 | 0.000723 |
| Chr 1 open reading frame | 1555226_s_at | BC008306 | −2.32 | −1.64 | −3.35 | 385.24 | 3.689 | 0.001641 |
| 43 | | | | | | | | |
| HSPA13 | 202558_s_at | NM_006948 | −2.38 | −1.62 | −3.69 | 227.34 | 3.56 | 0.002051 |
| CDNA FLJ13267 | 229713_at | AW665227 | −2.38 | −1.78 | −3.2 | 153.76 | 4.422 | 0.000354 |
| Chr 14 open reading | 212497_at | AI554879 | −2.44 | −1.6 | −3.94 | 167.26 | 3.288 | 0.003924 |
| frame 32 | | | | | | | | |
| sperm specific antigen 2 | 202506_at | NM_006751 | −2.52 | −1.7 | −3.65 | 286.31 | 3.307 | 0.004566 |
| epithelial membrane | 203729_at | NM_001425 | −2.57 | −1.74 | −4.01 | 1130.24 | 3.781 | 0.001283 |
| protein 3 | | | | | | | | |
| arrestin domain | 225283_at | AV701177 | −2.63 | −1.72 | −4.37 | 318.17 | 3.578 | 0.001999 |
| containing 4 | | | | | | | | |
| lymphocyte adaptor | 203320_at | NM_005475 | −2.84 | −1.96 | −3.99 | 961.09 | 3.808 | 0.001719 |
| protein | | | | | | | | |
| ring finger protein 125 | 207735_at | NM_017831 | −3.23 | −2.15 | −4.8 | 202.34 | 3.799 | 0.001752 |
| CREG1 | 201200_at | NM_003851 | −3.31 | −2.14 | −4.69 | 1177.93 | 3.378 | 0.004663 |
| dual specificity | 221563_at | N36770 | −3.35 | −1.94 | −6.89 | 868.23 | 3.367 | 0.003478 |
| phosphatase 10 | | | | | | | | |
| CD37 antigen | 204192_at | NM_001774 | −3.49 | −2.51 | −5.19 | 631.04 | 5.923 | 0.000013 |
| heat shock 90 kDa protein | 214359_s_at | AI218219 | −3.66 | −2.39 | −6.87 | 1551.01 | 5.572 | 0.000019 |
| 1, beta | | | | | | | | |
| HDGFRP3 | 216693_x_at | AL133102 | −4.78 | −2.9 | −7.96 | 148.97 | 3.805 | 0.001892 |
| HDGFRP3 | 209526_s_at | AB029156 | −6.72 | −3.83 | −11.8 | 161.97 | 3.721 | 0.002391 |
| HDGFRP3 | 209524_at | AK001280 | −9.53 | −4.67 | −30.43 | 339.46 | 3.736 | 0.002252 |

Supervised analysis of transcriptional profiles based on CRLF2 expression. The dataset included 22 adult B-ALL that lack characteristic gene rearrangements, divided by CRLF2 expression, and confirmed by CRLF2 qRT-PCR. The 8 cases with high CRLF2 expression were defined by a 105 gene expression signature (130 probe sets), using a p value cutoff of <0.005 and fold change (FC) ≧1.5.

A similar supervised approach was applied to identify differentially expressed genes based on CRLF2 expression in the pediatric B-ALL GEO datasets GSE11877 and GSE12995, and these were compared to the CRLF2 adult signature of 105 genes using Gene Set Enrichment Analysis (GSEA). Both pediatric signatures showed striking enrichment of the adult set (p<0.001).

To identify pathways that show differential expression in CRLF2-overexpressing B-ALL, GSEA for biological process gene groups from the Broad Institute's Molecular Signature Database were performed. Constituent genes of “JAK-STAT signaling” (p<0.001; false discovery rate (FDR)=0.012) and “cytokine-cytokine receptor interaction” (p<0.001; FDR=0) were significantly over-represented in CRLF2 overexpressing cases. Included among the genes enriched in the JAK-STAT signaling pathway were BCL-xL, PIM1, STAT5B and SOCS2.

In order to test the hypothesis that CRLF2 overexpression promotes a similar gene expression pattern to BCR/ABL, a BCR/ABL signature was obtained from the Oncomine® Concepts Map (http://www.oncomine.org), which identified the top up-regulated and down-regulated genes. Ross et al. (2003) Blood 102:2951-9. Using GSEA, the pediatric CRLF2 over-expression signatures from both datasets showed striking enrichment of the BCR/ABL-associated genes (p<0.001). Together, these findings establish common expression patterns between BCR/ABL-positive and CRLF2-overexpressing B-ALL in both children and adults.

wt CRLF2
SEQ ID NO: 1
MGRLVLLWGAAVFLLGGWMALGQGGAAEGVQIQIIYFNLETVQVTWNASKYS
RTNLTFHYRFNGDEAYDQCTNYLLQEGHTSGCLLDAEQRDDILYFSIRNGTHPV
FTASRWMVYYLKPSSPKHVRFSWHQDAVTVTCSDLSYGDLLYEVQYRSPFDTE
WQSKQENTCNVTIEGLDAEKCYSFWVRVKAMEDVYGPDTYPSDWSEVTCWQR
GEIRDACAETPTPPKPKLSKFILISSLAILLMVSLLLLSLWKLWRVKKFLIPSVPDP
KSIFPGLFEIHQGNFQEWITDTQNVAHLHKMAGAEQESGPEEPLVVQLAKTEAES
PRMLDPQTEEKEASGGSLQLPHQPLQGGDVVTIGGFTFVMNDRSYVAL
cDNA for wt CRLF2
SEQ ID NO: 2
aattcggcacgagggcatggggcggctggttctgctgtggggagctgccgtctttctgctgggaggctggatggctttggggc
aaggaggagcagcagaaggagtacagattcagatcatctacttcaatttagaaaccgtgcaggtgacatggaatgccagcaaa
tactccaggaccaacctgactttccactacagattcaacggtgatgaggcctatgaccagtgcaccaactaccactccaggaag
gtcacacttcggggtgcctcctagacgcagagcagcgagacgacattctctatttctccatcaggaatgggacgcaccccgtttt
caccgcaagtcgctggatggtttattacctgaaacccagttccccgaagcacgtgagattttcgtggcatcaggatgcagtgacg
gtgacgtgttctgacctgtcctacggggatctcctctatgaggttcagtaccggagccccttcgacaccgagtggcagtccaaac
aggaaaatacctgcaacgtcaccatagaaggcttggatgccgagaagtgttactctttctgggtcagggtgaaggctatggagg
atgtatatgggccagacacatacccaagcagactggtcagaggtgacatgctggcagagaggcgagattcgggatgcctgtgc
agagacaccaacgcctcccaaaccaaagctgtccaaatttattttaatttccagcctggccatccttctgatggtgtctctcctcctt
ctgtctttatggaaattatggagagtgaagaagtttctcattcccagcgtgccagacccgaaatccatcttccccgggctctttgag
atacaccaagggaacttccaggagtggatcacagacacccagaacgtggcccacctccacaagatggcaggtgcagagcaa
gaaagtggccccgaggagcccctggtagtccagttggccaagactgaagccgagtctcccaggatgctggacccacagacc
gaggagaaagaggcctctgggggatccctccagcttccccaccagcccctccaaggcggtgatgtggtcacaatcgggggct
tcacctttgtgatgaatgaccgctcctacgtggcgttgtgatggacacaccactgtcaaagtcaacgtcaggatccacgttgacat
ttaaagacagaggggactgtcccggggactccacaccaccatggatgggaagtctccacgccaatgatggtaggactaggag
actctgaagacccagcctcaccgcctaatgcggccactgccctgctaactttcccccacatgagtctctgtgttcaaaggcttgat
ggcagatgggagccaattgctccaggagatttactcccagttccttttcgtgcctgaacgttgtcacataaaccccaaggcagca
cgtccaaaatgctgtaaaaccatcttcccactctgtgagtccccagttccgtccatgtacctgttccatagcattggattctcggag
gattttttgtctgttttgagactccaaaccacctctacccctacaaaaaaaaaaaaaaaaaa
coding sequence (17-1132) from SEQ ID NO: 2
SEQ ID NO: 5
atggggcggctggttctgctgtggggagctgccgtctttctgctgggaggctggatggctttggggcaaggaggagcagcaga
aggagtacagattcagatcatctacttcaatttagaaaccgtgcaggtgacatggaatgccagcaaatactccaggaccaacctg
actttccactacagattcaacggtgatgaggcctatgaccagtgcaccaactaccttctccaggaaggtcacacttcggggtgcc
tcctagacgcagagcagcgagacgacattctctatttctccatcaggaatgggacgcaccccgttttcaccgcaagtcgctggat
ggtttattacctgaaacccagttccccgaagcacgtgagattttcgtggcatcaggatgcagtgacggtgacgtgttctgacctgt
cctacggggatctcctctatgaggttcagtaccggagccccttcgacaccgagtggcagtccaaacaggaaaatacctgcaac
gtcaccatagaaggcttggatgccgagaagtgttactctttctgggtcagggtgaaggctatggaggatgtatatgggccagac
acatacccaagcgactggtcagaggtgacatgctggcagagaggcgagattcgggatgcctgtgcagagacaccaacgcct
cccaaaccaaagctgtccaaatttattttaatttccagcctggccatccttctgatggtgtctctcctccttctgtctttatggaaattat
ggagagtgaagaagtttctcattcccagcgtgccagacccgaaatccatcttccccgggctctttgagatacaccaagggaactt
ccaggagtggatcacagacacccagaacgtggcccacctccacaagatggcaggtgcagagcaagaaagtggccccgagg
agcccctggtagtccagttggccaagactgaagccgagtctcccaggatgctggacccacagaccgaggagaaagaggcct
ctgggggatccctccagcttccccaccagcccctccaaggcggtgatgtggtcacaatcgggggcttcaccttgtgatgaatg
accgctcctacgtggcgttgtga
CRLF2 F232C
SEQ ID NO: 3
MGRLVLLWGAAVFLLGGWMALGQGGAAEGVQIQIIYFNLETVQVTWNASKYS
RTNLTFHYRFNGDEAYDQCTNYLLQEGHTSGCLLDAEQRDDILYFSIRNGTHPV
FTASRWMVYYLKPSSPKHVRFSWHQDAVTVTCSDLSYGDLLYEVQYRSPFDTE
WQSKQENTCNVTIEGLDAEKCYSFWVRVKAMEDVYGPDTYPSDWSEVTCWQR
GEIRDACAETPTPPKPKLSKCILISSLAILLMVSLLLLSLWKLWRVKKFLIPSVPDP
KSIFPGLFEIHQGNFQEWITDTQNVAHLHKMAGAEQESGPEEPLVVQLAKTEAES
PRMLDPQTEEKEASGGSLQLPHQPLQGGDVVTIGGFTFVMNDRSYVAL
coding sequence for CRLF2 F232C
SEQ ID NO: 4
atggggcggctggttctgctgtggggagctgccgtctttctgctgggaggctggatggctttggggcaaggaggagcagcaga
aggagtacagattcagatcatctacttcaatttagaaaccgtgcaggtgacatggaatgccagcaaatactccaggaccaacctg
actttccactacagattcaacggtgatgaggcctatgaccagtgcaccaactaccttctccaggaacctcacacttcggggtgcc
tcctagacgcagagcagcgagacgacattctctatttctccatcaggaatgggacgcaccccgttttcaccgcaagtcgctggat
ggtttattacctgaaacccagttccccgaagcacgtgagattttcgtggcatcaggatgcagtgacggtgacgtgttctgacctgt
cctacggggatctcctctatgaggttcagtaccggagccccttcgacaccgagtggcagtccaaacaggcccctacctgcaac
gtcaccatagaaggcttggatgccgagaagtgttactctttctgggtcagggtgaaggctatggaggatgtatatgggccagac
acatacccaagcgactggtcagaggtgacatgctggcagagaggcgagattcgggatgcctgtgcagagacaccaacgcct
cccaaaccaaagctgtccaaatgtattttaatttccagcctggccatccttctgatggtgtctctcctccttctgtctttatggaaattat
ggagagtgaagaagtttctcattcccagcgtgccagacccgaaatccatcttccccgggctctttgagatacaccaagggaactt
ccaggagtggatcacagacacccagaacgtggcccacctccacaagatggcaggtgcagagcaagaaagtggccccgagg
agcccctggtagtccagttggccaagactgaagccgagtctcccaggatgctggacccacagaccgaggagaaagaggcct
ctgggggatccctccagcttccccaccagcccctccaaggcggtgatgtggtcacaatcgggggcttcacctttgtgatgaatg
accgctcctacgtggcgttgtga

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. An isolated mutant human cytokine receptor-like factor 2 (CRLF2) polypeptide having an amino acid sequence at least 99% identical to SEQ ID NO:1 and comprising a F232C mutation.

2. An isolated nucleic acid molecule comprising a sequence that encodes the mutant human cytokine receptor-like factor 2 (CRLF2) polypeptide of claim 1.

3. A vector comprising the nucleic acid molecule of claim 2.

4. A cell comprising the vector of claim 3.

5. An isolated antibody, or antigen-binding fragment thereof, that binds specifically to the mutant human CRLF2 polypeptide of claim 1.

6. An isolated antibody, or antigen-binding fragment thereof, that binds specifically to a homodimeric protein comprising the mutant human CRLF2 polypeptide of claim 1.

7. The antibody of claim 5, wherein the antibody or antigen-binding fragment thereof is conjugated to a toxin.

8. A method of treating precursor B-cell acute lymphoblastic leukemia (B-ALL), comprising:
administering to a subject having B-ALL, wherein the B-ALL is characterized by mutant human cytokine receptor-like factor 2 (CRLF2) polypeptide having an amino acid sequence at least 99% identical to SEQ ID NO:1 and comprising a F232C mutation, an effective amount of an agent that inhibits signaling by the mutant CRLF2 to treat the B-ALL.

9. The method of claim 8, wherein the agent comprises an antibody or antigen-binding fragment thereof that binds specifically to the mutant human CRLF2 polypeptide.

10. The method of claim 9, wherein the antibody or antigen-binding fragment is conjugated to a toxin.

11. The method of claim 8, wherein the agent comprises an antisense oligonucleotide complementary to a polynucleotide encoding mutant human cytokine receptor-like factor 2 (CRLF2) polypeptide having an amino acid sequence at least 99% identical to SEQ ID NO:1 and comprising a F232C mutation.

12. The method of claim 8, wherein the agent comprises RNAi complementary to a polynucleotide encoding mutant human cytokine receptor-like factor 2 (CRLF2) polypeptide having an amino acid sequence at least 99% identical to SEQ ID NO:1 and comprising a F232C mutation.

13. The method of claim 8, further comprising administering to the subject an effective amount of a compound selected from the group consisting of JAK2 inhibitors, PKC inhibitors, HSP90 inhibitors, and any combination thereof.

14. A method for identifying a subject at increased risk of mortality from precursor B-cell acute lymphoblastic leukemia (B-ALL), comprising:
performing an assay on a sample isolated from a subject, wherein the assay detects presence of mutant human cytokine receptor-like factor 2 (CRLF2) characterized by an amino acid mutation F232C; and
identifying the subject as having increased risk of mortality from B-ALL when the performing the assay detects the presence of the mutant CRLF2 in the sample.

15. A method for identifying a subject at increased risk of mortality from precursor B-cell acute lymphoblastic leukemia (B-ALL), comprising:
determining the presence of mutant human cytokine receptor-like factor 2 (CRLF2) characterized by an amino acid mutation F232C by performing an assay on a sample isolated from a subject, wherein the presence of the mutant human CRLF2 in the sample indicates the subject is at increased risk of mortality from B-ALL.

16. The method of claim 14, wherein the subject has B-ALL.

17. The method of claim 15, wherein the subject has B-ALL.

18. The antibody of claim 6, wherein the antibody or antigen-binding fragment thereof is conjugated to a toxin.
